SEQUENCE LISTING (34 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 264 aa, Mus musculus
MKAVVLAVALVFLTGSQAWHVWQQDEPQSQWDKVKDFANVYVDAVKDSGRDYVSQFESSSLGQQLNLNLLENWDTLGSTVSQLQERLGPLTRDFWDNLEKETDWVRQEMNKDLEEVKQKVQPYLDEFQKKWKEDVELYRQKVAPLGAELQESARQKLQELQGRLSPVAEEFRDRMRTHVDSLRTQLAPHSEQMRESLAQRLAELKSNPTLNEYHTRAKTHLKTLGEKARPALEDLRHSLMPMLETLKTQVQSVIDKASETLTAQ

SEQ ID NO: 3 - protein, 148 aa, synthetic construct
  source 1..148: note = Derived from Homo sapiens
EVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 4 - protein, 150 aa, synthetic construct
  source 1..150: note = Derived from Mus musculus
EPHLGLWYFIAGAAPTTEELATFDPVDNIVFNMAAGSAPRQLQLRATIRTKSGVCVPRKWTYRLTEGKGNMELRTEGRPDMKTDLFSSSCPGGIMLKETGQGYQRFLLYNRSPHPPEKCVEEFKSLTSCLDFKAFLVTPRNQEACPLSSK

SEQ ID NO: 5 - protein, 168 aa, Homo sapiens
YQCPEHSQLTTLGVDGKEFPEVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 6 - protein, 170 aa, Mus musculus
NQCPEHSQLTALGMDDTETPEPHLGLWYFIAGAAPTTEELATFDPVDNIVFNMAAGSAPRQLQLRATIRTKSGVCVPRKWTYRLTEGKGNMELRTEGRPDMKTDLFSSSCPGGIMLKETGQGYQRFLLYNRSPHPPEKCVEEFKSLTSCLDFKAFLVTPRNQEACPLSSK

SEQ ID NO: 7 - protein, 20 aa, synthetic construct
  source 1..20: note = Derived from Homo sapiens
YQCPEHSQLTTLGVDGKEFP

SEQ ID NO: 8 - protein, 20 aa, synthetic construct
  source 1..20: note = Derived from Mus musculus
NQCPEHSQLTALGMDDTETP

SEQ ID NO: 9 - skipped

SEQ ID NO: 10 - protein, 4 aa, synthetic construct
SGGS

SEQ ID NO: 11 - protein, 60 aa, synthetic construct
  VARIANT 5..60: note = May be absent
SGGSSGGSSGGSSGGSSGGSSGGSSGGSSGGSSGGSSGGSSGGSSGGSSGGSSGGSSGGS

SEQ ID NO: 12 - protein, 60 aa, synthetic construct
  VARIANT 5..60: note = May be absent
GGGSGGGSGGGSGGGSGGGSGGGSGGGSGGGSGGGSGGGSGGGSGGGSGGGSGGGSGGGS

SEQ ID NO: 13 - protein, 75 aa, synthetic construct
  VARIANT 6..60: note = May be absent
GGGGSGGGGSGGGGSGGGGSGGGGSGGGGSGGGGSGGGGSGGGGSGGGGSGGGGSGGGGSGGGGSGGGGSGGGGS

SEQ ID NO: 14 - protein, 15 aa, synthetic construct
  VARIANT 2..15: note = May be absent
GGGGGGGGGGGGGGG

SEQ ID NO: 15 - protein, 75 aa, synthetic construct
  VARIANT 6..75: note = May be absent
EAAAKEAAAKEAAAKEAAAKEAAAKEAAAKEAAAKEAAAKEAAAKEAAAKEAAAKEAAAKEAAAKEAAAKEAAAK

SEQ ID NO: 16 - protein, 45 aa, synthetic construct
  VARIANT 4..45: note = May be absent
GGSGGSGGSGGSGGSGGSGGSGGSGGSGGSGGSGGSGGSGGSGGS

SEQ ID NO: 17 - protein, 16 aa, synthetic construct
SGSETPGTSESATPES

SEQ ID NO: 18 - protein, 30 aa, synthetic construct
  VARIANT 3..30: note = May be absent
XPXPXPXPXPXPXPXPXPXPXPXPXPXPXP

SEQ ID NO: 19 - protein, 24 aa, synthetic construct
SGGSSGSETPGTSESATPESSGGS

SEQ ID NO: 20 - protein, 32 aa, synthetic construct
SGGSSGGSSGSETPGTSESATPESSGGSSGGS

SEQ ID NO: 21 - protein, 104 aa, synthetic construct
GGSGGSPGSPAGSPTSTEEGTSESATPESGPGTSTEPSEGSAPGSPAGSPTSTEEGTSTEPSEGSAPGTSTEPSEGSAPGTSESATPESGPGSEPATSGGSGGS

SEQ ID NO: 22 - protein, 5 aa, synthetic construct
GGGGS

SEQ ID NO: 23 - protein, 451 aa, synthetic construct
MKAVVLAVALVFLTGSQAWHVWQQDEPQSQWDKVKDFANVYVDAVKDSGRDYVSQFESSSLGQQLNLNLLENWDTLGSTVSQLQERLGPLTRDFWDNLEKETDWVRQEMNKDLEEVKQKVQPYLDEFQKKWKEDVELYRQKVAPLGAELQESARQKLQELQGRLSPVAEEFRDRMRTHVDSLRTQLAPHSEQMRESLAQRLAELKSNPTLNEYHTRAKTHLKTLGEKARPALEDLRHSLMPMLETLKTKAQSVIDKASETLTAQTRTRPLGGGGSNQCPEHSQLTALGMDDTETPEPHLGLWYFIAGAASTTEELATFDPVDNIVFNMAAGSAPRQLQLRATIRTKSGVCVPRKWTYRLTEGKGNMELRTEGRPDMKTDLFSSSCPGGIMLKETGQGYQRFLLYNRSPHPPEKCVEEFQSLTSCLDFKAFLVTPRNQEACPLSSKHHHHHH

SEQ ID NO: 24 - protein, 445 aa, synthetic construct
MKAVVLAVALVFLTGSQAWHVWQQDEPQSQWDKVKDFANVYVDAVKDSGRDYVSQFESSSLGQQLNLNLLENWDTLGSTVSQLQERLGPLTRDFWDNLEKETDWVRQEMNKDLEEVKQKVQPYLDEFQKKWKEDVELYRQKVAPLGAELQESARQKLQELQGRLSPVAEEFRDRMRTHVDSLRTQLAPHSEQMRESLAQRLAELKSNPTLNEYHTRAKTHLKTLGEKARPALEDLRHSLMPMLETLKTKAQSVIDKASETLTAQTRTRPLGGGGSNQCPEHSQLTALGMDDTETPEPHLGLWYFIAGAASTTEELATFDPVDNIVFNMAAGSAPRQLQLRATIRTKSGVCVPRKWTYRLTEGKGNMELRTEGRPDMKTDLFSSSCPGGIMLKETGQGYQRFLLYNRSPHPPEKCVEEFQSLTSCLDFKAFLVTPRNQEACPLSSK

SEQ ID NO: 25 - DNA, 1357 bp, synthetic construct
  source 1..1357: mol_type = other DNA
atgaaagctgtggtgctggccgtggctctggtcttcctgacagggagccaggcttggcacgtatggcagcaagatgaaccccagtcccaatgggacaaagtgaaggatttcgctaatgtgtatgtggatgcggtcaaagacagcggcagagactatgtgtcccagtttgaatcctcctccttgggccaacagctgaacctgaatctcctggaaaactgggacactctgggttcaaccgttagtcagctgcaggaacggctgggcccattgactcgggacttctgggataacctggagaaagaaacagattgggtgagacaggagatgaacaaggacctagaggaagtgaaacagaaggtgcagccctacctggacgaattccagaagaaatggaaagaggatgtggagctctaccgccagaaggtggcgcctctgggcgccgagctgcaggagagcgcgcgccagaagctgcaggagctgcaagggagactgtcccctgtggctgaggaatttcgcgaccgccatgcgcacacacgtagactctctgcgcacacagctagcgccccacagcgaacagatgcgcgagagcctggcccagcgcctggctgagctcaagagcaaccctaccttgaacgagtaccacaccagggccaaaacccacctgaagacacttggcgagaaagccagacctgcgctggaggacctgcgccatagtctgatgcccatgctggagacgcttaagaccaaagcccagagtgtgatcgacaaggccagcgagactctgactgcccagacgcgtacgcggccgcttggtggaggtggatctaatcagtgccctgagcacagtcaactaactgcgctgggaatggacgacacagagaccccagagccccacctgggcctgtggtactttattgcgggagcagcctccaccacggaagagttggcaacttttgatccggtggacaatattgtcttcaacatggctgccggctctgccccaaggcagctccagcttcgtgctaccatccgcacgaaaagtggggtctgtgtgccccggaagtggacataccgattgactgaagggaaaggaaacatggaactcagaactgaagggcgcccagacatgaaaacagacctgttctccagctcgtgcccaggaggaatcatgctgaaagagacgggccagggctaccagcgctttctcctctacaatcggtcaccacaccctccagagaagtgtgtggaggaattccagtctctgacctcttgcttggacttcaaagccttcttagtgactcccaggaatcaagaggcctgcccgctgtccagcaagcatcaccatcaccatcactga

SEQ ID NO: 26 - DNA, 1339 bp, synthetic construct
  source 1..1339: mol_type = other DNA
atgaaagctgtggtgctggccgtggctctggtcttcctgacagggagccaggcttggcacgtatggcagcaagatgaaccccagtcccaatgggacaaagtgaaggatttcgctaatgtgtatgtggatgcggtcaaagacagcggcagagactatgtgtcccagtttgaatcctcctccttgggccaacagctgaacctgaatctcctggaaaactgggacactctgggttcaaccgttagtcagctgcaggaacggctgggcccattgactcgggacttctgggataacctggagaaagaaacagattgggtgagacaggagatgaacaaggacctagaggaagtgaaacagaaggtgcagccctacctggacgaattccagaagaaatggaaagaggatgtggagctctaccgccagaaggtggcgcctctgggcgccgagctgcaggagagcgcgcgccagaagctgcaggagctgcaagggagactgtcccctgtggctgaggaatttcgcgaccgccatgcgcacacacgtagactctctgcgcacacagctagcgccccacagcgaacagatgcgcgagagcctggcccagcgcctggctgagctcaagagcaaccctaccttgaacgagtaccacaccagggccaaaacccacctgaagacacttggcgagaaagccagacctgcgctggaggacctgcgccatagtctgatgcccatgctggagacgcttaagaccaaagcccagagtgtgatcgacaaggccagcgagactctgactgcccagacgcgtacgcggccgcttggtggaggtggatctaatcagtgccctgagcacagtcaactaactgcgctgggaatggacgacacagagaccccagagccccacctgggcctgtggtactttattgcgggagcagcctccaccacggaagagttggcaacttttgatccggtggacaatattgtcttcaacatggctgccggctctgccccaaggcagctccagcttcgtgctaccatccgcacgaaaagtggggtctgtgtgccccggaagtggacataccgattgactgaagggaaaggaaacatggaactcagaactgaagggcgcccagacatgaaaacagacctgttctccagctcgtgcccaggaggaatcatgctgaaagagacgggccagggctaccagcgctttctcctctacaatcggtcaccacaccctccagagaagtgtgtggaggaattccagtctctgacctcttgcttggacttcaaagccttcttagtgactcccaggaatcaagaggcctgcccgctgtccagcaagtga

SEQ ID NO: 27 - protein, 264 aa, synthetic construct
MKAVVLAVALVFLTGSQAWHVWQQDEPQSQWDKVKDFANVYVDAVKDSGRDYVSQFESSSLGQQLNLNLLENWDTLGSTVSQLQERLGPLTRDFWDNLEKETDWVRQEMNKDLEEVKQKVQPYLDEFQKKWKEDVELYRQKVAPLGAELQESARQKLQELQGRLSPVAEEFRDRMRTHVDSLRTQLAPHSEQMRESLAQRLAELKSNPTLNEYHTRAKTHLKTLGEKARPALEDLRHSLMPMLETLKTKAQSVIDKASETLTAQ

SEQ ID NO: 28 - protein, 170 aa, synthetic construct
NQCPEHSQLTALGMDDTETPEPHLGLWYFIAGAASTTEELATFDPVDNIVFNMAAGSAPRQLQLRATIRTKSGVCVPRKWTYRLTEGKGNMELRTEGRPDMKTDLFSSSCPGGIMLKETGQGYQRFLLYNRSPHPPEKCVEEFQSLTSCLDFKAFLVTPRNQEACPLSSK

SEQ ID NO: 29 - DNA, 32 bp, synthetic construct
  source 1..32: mol_type = other DNA
ttttctagagaggagatctgccgccgcgatcg

SEQ ID NO: 30 - DNA, 31 bp, synthetic construct
  source 1..31: mol_type = other DNA
tttgcggccgcgtacgtctgggcagtcagag

SEQ ID NO: 31 - DNA, 36 bp, synthetic construct
  source 1..36: mol_type = other DNA
ggtggaggtggatctaatcagtgccctgagcacagt

SEQ ID NO: 32 - DNA, 33 bp, synthetic construct
  source 1..33: mol_type = other DNA
gatggtgatgtcccttgctggacagcgggcagg

SEQ ID NO: 33 - DNA, 36 bp, synthetic construct
  source 1..36: mol_type = other DNA
tttgcggccgcttggtggaggtggatctaatcagtg

SEQ ID NO: 34 - DNA, 41 bp, synthetic construct
  source 1..41: mol_type = other DNA
tttggatcctcagtgatggtgatggtgatgtcccttgctgg

(19) United States
(12) Patent Application Publication
(10) Pub. No.: US 2025/0042974 A1
(43) Pub. Date: Feb. 6, 2025

(54) LIPID MEDIATOR CHAPERONE AND USES THEREOF

(71) Applicant: The Children's Medical Center Corporation, Boston, MA (US)

(72) Inventors: Timothy T. Hla, Wellesley, MA (US); Steven L. Swendeman, Cambridge, MA (US)

(73) Assignee: The Children's Medical Center Corporation, Boston, MA (US)

(21) Appl. No.: 18/697,295

(22) PCT Filed: Sep. 30, 2022
(86) PCT No.: PCT/US2022/077391

§ 371 Date: Mar. 29, 2024

Related U.S. Application Data
(60) Provisional application No. 63/251,457, filed on Oct. 1, 2021.

(51) Int. Cl. C07K 14/775; A61K 38/00; C12N 15/79

(52) U.S. Cl. CPC C07K 14/775; C12N 15/79; A61K 38/00; C07K 2319/30

(57) ABSTRACT

Described herein are a ApoA1 and ApoM (A1M) fusion proteins, lipidated A1M fusion proteins, nanoparticles comprising AM1 fusion proteins, and methods of use thereof for treatment of vascular and inflammatory disorders.

### RELATED APPLICATION

This application claims the benefit under 35 U.S.C. § 119(e) to U.S. Provisional Application No. 63/251,457 filed on Oct. 1, 2021 and entitled “LIPID MEDIATOR CHAPERONE FOR TREATMENT OF VASCULAR AND INFLAMMATORY DISORDERS,” the entire contents of which are incorporated herein by reference.

### FEDERALLY SPONSORED RESEARCH

This invention was made with government support under grant number H1135821, awarded by the national institutes of health. The government has certain rights in the invention.

### REFERENCE TO AN ELECTRONIC SEQUENCE LISTING

The contents of the electronic sequence listing (C123370227WO00-SEQ-RE.xml; Size: 38,925 bytes; and Date of Creation: Sep. 30, 2022) is herein incorporated by reference in its entirety.

### BACKGROUND

Lipid mediators are derivatives of cellular fatty molecules that act on specific cell-surface receptors to induce biological effects. Well known examples include prostaglandins, sphingosine 1-phosphate (S1P), leukotrienes, etc. Due to their lipophilicity, they are associated with protein molecules to help them diffuse in the extracellular environment and bind to receptors. Therefore, lipid mediator chaperones (LMC) are defined as protein molecules that bind to lipid mediators, control their stability and help in the activation of receptors to induce cellular responses.

### SUMMARY

Endothelial cell function is essential for normal cardiovascular homeostasis. Many environmental and intrinsic risk factors for cardiovascular and cerebrovascular diseases cause endothelial dysfunction. Indeed, dysfunctional endothelium is implicated in the development of vascular diseases (e.g., as described in Girouard et al., Journal of Applied Physiology 100, 328-335, 2006, incorporated herein by reference). On the other hand, various endogenous factors promote optimal endothelial function and counteract the risk factors (e.g., as described in Libby et al., Journal of the American College of Cardiology 54, 2129-2138, 2009, incorporated herein by reference). Among such factors is the high-density lipoprotein (HDL), a multifunctional circulating nanoparticle. Plasma HDL concentrations are shown to be correlated with reduced risk from cardiovascular and cerebrovascular diseases (e.g., as described in Libby et al., Journal of the American College of Cardiology 54, 2129-2138, 2009, incorporated herein by reference) as well as improved outcomes after an ischemic event (e.g., as described in Makihara et al., Cerebrovascular Diseases 33, 240-247, 2012; and Olsson et al., European Heart Journal 26, 890-896, 2005, incorporated herein by reference). HDL particles are heterogeneous, contain numerous bioactive factors and regulate vascular, metabolic and immune functions, suggesting that specific HDL particle subtypes regulate unique functions in the cardiovascular system.

The present disclosure, in some aspects, relates to a multifunctional lipid mediator chaperone called ApoA1-ApoM (A1M) and methods of use thereof. A1M is a recombinant fusion protein, which consists of the HDL-associated proteins Apolipoprotein A1 (ApoA1) and Apolipoprotein M (ApoM) and can act as a chaperone for multiple biologically active lipids. In addition, it forms HDL-like lipoprotein particles that carry lipid mediators such as S1P and prostacyclin (PGI2). The S1P- and PGI2-bound A1M nanodiscs protect the vascular endothelial cells and inhibit thromboinflammatory responses.

Many acute and chronic human pathologies initiate with dysfunction of endothelium in vascular beds. The mechanical, metabolic, or pathogen driven endothelial dysfunction leads to induction of inflammatory mediators and cellular receptors, which promote platelet driven thrombosis and activation of the innate immune system, resulting in subsequent amplification of the inflammatory response, destruction of vascular beds, and pathology in major organ systems. Thus, the potential application of ApoA1-ApoM-S1P/Iloprost is far ranging including viral or bacterial infections and traumatic or surgical injuries where extensive thrombosis is an indication, and clinical episodes involving vascular-associated thrombotic inflammation in cardiovascular diseases, cerebrovascular diseases, diabetes, autoimmune syndromes and chronic inflammatory diseases. Thus, A1M is designed to provide a three-pronged therapeutic to (A) reduce both inflammation in endothelium and innate immune cell amplification of inflammation; (B) inhibit pathogenic platelet-driven thrombosis as A1M-Iloprost; and (C) provide protection of endothelial barrier function and maintain vascular homeostasis as A1M-S1P.

In some aspects, the present disclosure relates to a fusion protein comprising ApoA1 and ApoM. In some embodiments, ApoA1 comprises an amino acid sequence that is 90% identical to any one of SEQ ID NOs: 1-2, optionally wherein the ApoA1 comprises the amino acid sequence any one of SEQ ID NOs: 1-2. In some embodiments, ApoM comprises an amino acid sequence that is 90% identical to any one of SEQ ID NOs: 3-4, optionally wherein the ApoA1 comprises the amino acid sequence of any one of SEQ ID NOs: 3-4. In some embodiments, the ApoA1 is fused to the N-terminus of ApoM. In some embodiments, the ApoA1 is fused to the C-terminus of ApoM. In some embodiments, the ApoA1 and ApoM are fused via a linker, optionally wherein the linker is a peptide linker. In some embodiments, the linker comprises the amino acid sequence of SEQ ID NO: 22. In some embodiments, the fusion protein comprises an amino acid sequence that is 90% identical to any one of SEQ ID NOs: 23-24, optionally wherein the fusion protein comprises the amino acid sequence of any one of SEQ ID NOs: 23-24.

In some aspects, the present disclosure related to a nucleic acid molecule comprising a polynucleotide sequence encoding the fusion protein as described above. In some embodiments, the polynucleotide sequence comprises a nucleotide sequence that is at least 90% identical to SEQ ID NO: 25 or 26, optionally wherein the polynucleotide sequence comprises the nucleotide sequence of SEQ ID NO: 25 or 26. In some embodiments, the polynucleotide sequence is operably linked to a promoter. In some embodiments, a construct comprises the nucleic acid molecule described above. In some embodiments, the construct is a plasmid or vector. In some embodiments, the construct is a viral vector.

In some aspects, the present disclosure related to a cell comprising the fusion protein as described above, the nucleic acid sequence as described above, or the construct as described above. In some embodiments, the cell is a prokaryotic cell. In some embodiments, the cell is a eukaryotic cell, optionally a human cell.

In some embodiments, a lipoprotein comprises the fusion protein as described above and a lipid. In some embodiments, the lipid is a S1P receptor agonist or antagonist, or a prostaglandin agonist or antagonist. In some embodiments, the lipid is selected from the group consisting of a prostaglandin, sphingosine 1-phosphate (S1P), a leukotriene, phosphatidyl choline. In some embodiments, the lipid is Iloprost. In some embodiments, the lipid is sphingosine-1-phosphate. In some embodiments, the lipoprotein is non-covalently bound to the lipid. In some embodiments, the lipoprotein is covalently bound to the lipid. In some embodiments, the lipoprotein is incorporated into a nanoparticle. In some embodiments, the nanoparticle is a nanodisk. In some embodiments, the nanoparticle is 70% unlipidated fusion protein and 30% lipoprotein.

In some aspects, the present disclosure relates to a method of treating a subject having a disease associated with vascular endothelial dysfunction, comprising administering the fusion protein of any as described above or the lipoprotein as described above.

In some embodiments, the disease is selected from the group consisting of thrombosis, or thrombotic inflammation. In some embodiments, the thrombotic inflammation is associated with cardiovascular disease, cerebrovascular disease, diabetes, atherosclerosis, an autoimmune syndrome or chronic inflammatory diseases.

In some aspects, the present disclosure relates to a method of reducing inflammation in a subject, comprising administering a drug selected from the group consisting of the fusion protein as described above, or the lipoprotein as described above. In some embodiments, the drug is administered in a therapeutically effective amount. In some embodiments, the inflammation is associated with TNFalpha-induced NF-kappaB activation. In some embodiments, the inflammation is associated with cardiovascular disease, cerebrovascular disease, diabetes, atherosclerosis, an autoimmune syndrome or chronic inflammatory diseases.

In some embodiments, the disease is diabetic nephropathy, lupus, and COVID-19 syndrome induced blood clots. In some embodiments, the method further comprises administrating an anti-thrombin agent. In some embodiments, the anti-thrombin agent is Angiopoietin1 or Activated Protein C (APC).

### BRIEF DESCRIPTION OF DRAWINGS

FIGS. 1A-1E show production, purification, and characterization of S1P binding by the ApoA1-ApoM fusion protein. (FIG. 1A) Representation of potential model structure of S1P bound to ApoA1-ApoM based on previously suggested models of ApoA1 in a lipidated state and the determined crystal structure of ApoM. (FIG. 1B) CHO-S cell derived, nickel-bead purified ApoA1-ApoM (4 μg) was analyzed by reducing 10% SDS_PAGE and Stained with Coomassie Brilliant Blue. (FIG. 1C) Purified ApoA1-ApoM (100 μg) was incubated or not with S1P for 24 hours, purified by gel filtration chromatography and analyzed for sphingolipids by electrospray ionization-MS/MS. The resulting data are the means+/−SD; N=5 independent experiments. (FIGS. 1D, 1E) FPLC Elution profile of recombinant ApoA1-ApoM. FPLC elution Plasma standards (see FIG. 8): LDL Lipoprotein (22-26 ml) HDL lipoprotein (28-32 ml), Soluble protein fraction (32-38 ml). (FIG. 1D) OD280 trace of nascent ApoA1-ApoM protein (100 μg): minor peak (31.5 ml) and major peak (35.5 ml) (FIG. 1E) lipidated A1M-S1P (1 mg) (Peak 32 ml).

FIGS. 2A-2F show A1M-S1P activates S1PRs. Nanobit analysis was performed to analyze of S1PR1 signal-dependent activation of Gi□ (FIG. 2A) and S1PR1 receptor coupling to β-arrestin (FIG. 2B). Albumin-S1P, ApoM-Fc-S1P, and ApoA1-ApoM-S1P were assayed by titration analysis over 4-logs of ligand concentration (0.32 nM, 1 nM, 3.2 nM, 10 nM, 32 nM, 100 nM, 320 nM, 1 μM S1P). N=3 independent experiments. Temporal analysis of maximal ligand-dependent receptor activation (1 μM) of Giα (FIG. 2C) and β-arrestin (FIG. 2D) Data were analyzed as ((relative luciferase activity−baseline/maximal signal−baseline)*100). (N=3 separate experiments). Nanobit analysis was performed to analyze S1PR2 and S1PR3 receptor coupling to β-arrestin (FIGS. 2E, 2F) using the same titration as in (FIG. 2B.) N=3 independent experiments.

FIGS. 3A-3H show ApoA1-ApoM-S1P maintains endothelial barrier function in vitro and co-protects endothelial barrier in response to Thrombin-induced barrier degradation. (FIG. 3A) HUVECs were analyzed for A1M-S1P-dependent enhancement of barrier function in a dose response analysis by real-time measurement of TEER using 0.8, 2.4, 8, 24 μg/ml or 10, 30, 100, and 300 nM S1P or 24 μg of A1M. (FIG. 3B) Comparison of A1M-S1P and ApoM-Fc-S1P by TEER using ApoA1-ApoM-S1P (1, 2, 4, 8, or 16 μg/ml; 12.5, 25, 50, 100, or 200 nM S1P), ApoA1-ApoM (1, 2, 4, 8, or 16 μg/ml), ApoM-Fc (1, 2, 4, 8, or 16 μg/ml; 12.5, 25, 50, 100, or 200 nM S1P), or ApoM-Fc (1, 2, 4, 8, or 16 μg/ml). All data were compared to baseline vehicle treatment and presented as area under the curve (N=3 independent experiments; expressed as mean±SD. P<0.0001, two-way ANOVA followed by t test for S1P containing chaperones vs S1P-negative controls). (FIG. 3C) HUVECs were analyzed for barrier protection by TEER analysis using ApoM-S1P (30 nM) and Angiopoietin1 (300 ng/ml) individually or in combination. (FIG. 3D) HUVECs were analyzed for barrier protection by TEER analysis in response to Thrombin (1 U/ml) treatment using ApoM-S1P (200 nM), Angiopoietin1 (300 ng/ml) or in combination. For FIGS. 3C and 3D, data were analyzed by non-parametric t-test (Mann-Whitney) of control or individual treatments vs combined treatments (****P<0.0001). (FIG. 3E) HUVECs were analyzed for barrier protection by TEER analysis using either A1M-S1P (30 nM) (FIG. 3E) or ApoMFc-S1P (30 nM) (FIG. 3G) in conjunction with APC (5 μg/ml). After 1 hour pretreatment with inhibitors, Thrombin was added for an additional 2 hours. Efficacy was determined for both assays by both analysis of individual traces or Area Under the Curve analysis (n=3) followed by non-parametric t-test (Mann-Whitney) of control or individual treatments vs combined treatments (FIGS. 3F, 3G). ****P<0.0001 for (FIGS. 3E, 3G) and P<0.01 for (FIGS. 3F, 3G). FIG. 3F and FIG. 3H show graphs of the relative resistance (Area Under the Curve, “A.U.C.”) of different treatment conditions.

FIGS. 4A-4C show A1M attenuates TNFα-dependent Inflammation. (FIG. 4A) HMEC NF-κB-luciferase reporter cells were assayed for TNFα-induced NF-κB-dependent luciferase activity. A dose response was performed using ApoA1, ApoA1-ApoM and ApoA1-ApoM-S1P (25, 50, 100, 200 and 400 μg/ml). Data are presented as the mean of 3 independent experiments +/−S.D. Statistical analysis using student t-test gave a **P<0.01 for all data compared to TNFα and *P<0.05 for ApoA1-Apom or ApoA1-ApoM-S1P vs ApoA1 alone. (FIG. 4B) A dose response was performed using ApoM-Fc and Albumin-S1P (25, 50, 100, 200 and 400 μg/ml). Data are presented as the mean of 3 independent experiments+/−S.D. (FIG. 4C) HUVEC were assayed for TNFα induction of ICAM-1 expression by western blot analysis. Cultures were pre-treated for 10 minutes with ApomFc-S1P (100 nM), Iloprost (200 nM), or both in combination, as well as A1M, A1M-S1P, A1M-Iloprost (all 200 μg/ml) and induced with TNFα (10 ng/ml) for 5 hours. Image J analysis was performed comparing ICAM1/Actin expression.

FIGS. 5A-5F show production of A1M-Iloprost and TEER analysis of A1M-S1P and A1M-Iloprost. (FIG. 5A) OD280 trace of 1 mg of A1M lipidated with PC and Iloprost (see methods) and purified by FPLC. Fraction elution profile is compared to established fractionation of mouse plasma (See FIGS. 1A-1E and FIG. 8.). (FIG. 5B) CREB-luciferase reporter assay for detecting Iloprost function. The reporter cell line was stimulated with vehicle or A1M-Iloprost (1, 2, 4, 8, 16, or 32 μg/ml) for 8 hours and cell lysates were assayed for Luciferase activity. (FIG. 5C) HUVECs were analyzed for S1P-dependent enhancement of barrier function in a dose-response analysis by real-time measurement of TEER. At time 0, either vehicle or ApoA1-ApoM-S1P (8 μg/ml; 100 nM S1P), or ApoA1-ApoM-Iloprost (25 μg/ml; 200 nM Iloprost), or both treatments were added. All data was compared to baseline vehicle treatment and the area under the curve was determined (FIG. 5D) (N=4 independent experiments; expressed as mean±SEM. ****P<0.0001,*P=0.05 based on two-way ANOVA followed by non-parametric t-test (Mann-Whitney) of control or individual treatments vs combined treatments). TEER analysis of HDL-S1P (100 Nm S1P) (FIG. 5E) and the S1PR1 agonist AUY954 (1 μM) (FIG. 5F) alone, and in combination with Iloprost (200 nM) (N=3 or more).

FIGS. 6A-6D show ApoA1-Iloprost inhibits Human platelet Aggregation and ApoM-S1P and ApoA1-Iloprost inhibit fMLP-dependent ROS activation in Neutrophils. Isolated human platelets were assayed for aggregation in response to the thrombin peptide SFLLRN. Presented are the results for replicate analyses for vehicle control, ApoA1-ApoMIloprost (10 nM drug), ApoA1-ApoM-S1P (S1P 200 nM), or in combination (FIG. 6A). A narrow-range dose response was performed using either free Iloprost or equivalent ApoA1-Iloprost (2, 3, 4, 5, 6, 10, 20 nM drug). N=2 independent experiments (FIG. 6B) EC₅₀ Quantification for free Iloprost (˜11 nM) and ApoA1 nanodisc associated Illoprost (˜9 nM) suggest retention of Iloprose drug potency in nanodiscs. (FIG. 6C) Isolated peritoneal Mouse Neutrophils were assayed for ROS expression after fMLP stimulation. Cells were pre-incubated for 10 minutes with vehicle control, ApoM-S1P (S1P 100 nM), ApoA1-Iloprost (160 nM drug), or in combination. Data are presented as direct fluorescence after background subtraction before stimulation (FIG. 6D) Composite data for N=4 independent experiments. Data are presented as Area under the Curve followed by normalization to fMLP stimulation alone and are expressed as a percent of untreated signal. Data were analyzed by non-parametric t-test (Mann-Whitney) and P values<0.0001 were obtained for ApoM-S1P, ApoA1-Iloprost, or in combination versus Fmlp stimulation.

FIGS. 7A-7B show the nucleotide (SEQ ID NO: 25) and amino acid sequence (SEQ ID NO: 23) of ApoA1-ApoM identifying the features of the fusion protein. The ApoA1-ApoM fusion contains the signal peptide of native murine ApoA1 and amino acids 1-264 of the open reading frame followed by one (Gly4Ser1) flexible linker region, amino acids 21-190 of murine ApoM, a 6× histidine purification tag, and a stop translation codon.

FIG. 8 shows the FPLC trace of normal mouse plasma. 150 μl mouse plasma, previously clarified by centrifugation at 20,000×g for 20 minutes, was injected for FPLC analysis (See methods). 0.4 ml fractions were collected from the sample and fractions were analyzed by SDS-PAGE using both Coomassie Blue staining and western blot analysis to confirm fraction identity. Presented is the OD280 trace of the analysis.

FIGS. 9A-9B show titration assays for Thrombin inhibition by TEER analysis. (FIG. 9A) HUVECs were analyzed for barrier protection by TEER analysis (n=3) for 2 hours using ApoM-S1P (1, 3, 10, 30, and 100 nM S1P) in combination with Thrombin (1 U/ML). (FIG. 9B) HUVECs were analyzed for barrier protection by TEER analysis (n=3) for 2 hours using Activated Protein C (APC; 1, 5, 20, and 50 μg/ml) in combination with Thrombin (1 U/ml).

FIG. 10 shows FPLC trace of purification of ApoA1-Iloprost. 0.5 mg of human ApoA1 (Sigma) was lipidated as described (See methods) 0.4 ml fractions were collected from the sample.

### DETAILED DESCRIPTION

The present disclosure relates to a multifunctional lipid mediator chaperone called ApoA1-ApoM (A1M) and methods of use thereof. A1M is a recombinant fusion protein, which consists of the HDL-associated proteins Apolipoprotein A1 (ApoA1) and Apolipoprotein M (ApoM) and can act as a chaperone for multiple biologically active lipids.

Apolipoprotein A1 (ApoA1) is a 28-kDa protein that is the major structural protein of high-density lipoprotein (HDL). In some embodiments, ApoA1 comprises an amino acid sequence that is at least 70% identical to any one of SEQ ID NOs: 1-2 or 27. For example, the ApoA1 may comprise an amino acid sequence that is at least 70%, at least 80%, at least 90%, at least 95%, or at least 99% identical to any one of SEQ ID NOs: 1-2 or 27. In some embodiments, ApoA1 comprises an amino acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1-2 or 27. In some embodiments, ApoA1 comprises the amino acid sequence of any one of SEQ ID NOs: 1-2 or 27. In some embodiments, ApoA1 consists of the amino acid sequence of any one of SEQ ID NOs: 1-2 or 27. In some embodiments, ApoA1 protein does not comprise a signal peptide sequence.

“Apolipoprotein M (ApoM)” is a 26-kDa protein that is mainly associated with high-density lipoprotein (HDL) in mammalian (e.g., human) plasma, with a small proportion present in triglyceride-rich lipoproteins (TGRLP) and low-density lipoproteins (LDL). It belongs to lipocalin protein superfamily. ApoM is only expressed in liver and in kidney and small amounts are found in fetal liver and kidney. Expression of native ApoM could be regulated by platelet activating factor (PAF), transforming growth factors (TGF), insulin-like growth factor (IGF) and leptin in vivo and/or in vitro. The ApoM may be from any mammal (e.g., a human or a murine such as a mouse or a rat). The amino acid sequences of wild-type human and mouse ApoM, and nucleotide sequences encoding such are provided in Table 1. It is to be understood that the sequences provided are for illustration purpose only and are not meant to be limiting. The ApoM moiety inherently acts as a chaperone for the biologically active sphingolipid, Sphingosine-1-Phosphate (S1P), which has clearly demonstrated vascular protective properties including endothelial homeostasis and enhancement of endothelial barrier function (reduction of leakiness) in vitro and in vivo.

In some embodiments, ApoM comprises an amino acid sequence that is at least 70% identical to any one of SEQ ID NOs: 3-6 or 28. For example, ApoM may comprise an amino acid sequence that is at least 70%, at least 80%, at least 90%, at least 95%, or at least 99% identical to any one of SEQ ID NOs: 3-6 or 28. In some embodiments, ApoM comprises an amino acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 3-6 or 28. In some embodiments, ApoM comprises the amino acid sequence of to any one of SEQ ID NOs: 3-6 or 28. In some embodiments, ApoM consists of the amino acid sequence of any one of SEQ ID NOs: 3-6 or 28. In some embodiments, ApoM protein does not comprise a signal peptide sequence. In some embodiments, ApoM protein comprises a signal peptide sequence.

### ApoA1-ApoM1 Fusion Proteins

In some aspects, the present disclosure relates to a fusion protein comprising ApoA1 and ApoM.

A “fusion protein” as used herein, refers to a hybrid polypeptide which comprises protein domains from at least two different proteins (e.g. A1M). One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A fusion protein may comprise different domains, for example, an ApoM domain and ApoA1 domain. In some embodiments, the ApoM is fused at the N-terminus of the ApoA1. In some embodiments, the ApoM is fused at the C-terminus of the ApoA1.

In some embodiments, A1M comprises an amino acid sequence that is at least 70% identical to any one of SEQ ID NOs: 23-24. For example, A1M may comprise an amino acid sequence that is at least 70%, at least 80%, at least 90%, at least 95%, or at least 99% identical to any one of SEQ ID NOs: 23-24. In some embodiments, A1M comprises an amino acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 23-24. In some embodiments, A1M comprises the amino acid sequence of to any one of SEQ ID NOs: 23-24. In some embodiments, A1M consists of the amino acid sequence of any one of SEQ ID NOs: 23-24.

### Linkers

In some embodiments, the ApoM and ApoA1 are fused via a linker. A “linker” refers to a chemical group or a molecule linking two molecules or moieties, e.g., two domains of a fusion protein. Typically, the linker is positioned between, or flanked by, two groups, molecules, domains, or other moieties and connected to each one via a covalent bond, thus connecting the two. The linker may be as simple as a covalent bond, or it may be a polymeric linker many atoms in length. In some embodiments, the linker is a polypeptide or based on amino acids. In some embodiments, the linker is not peptide-like. In some embodiments, the linker is a covalent bond (e.g., a carbon-carbon bond, disulfide bond, carbon-heteroatom bond, etc.). In some embodiments, the linker is a carbon-nitrogen bond of an amide linkage. In some embodiments, the linker is a cyclic or acyclic, substituted or unsubstituted, branched or unbranched aliphatic or heteroaliphatic linker. In some embodiments, the linker is polymeric (e.g., polyethylene, polyethylene glycol, polyamide, polyester, etc.). In some embodiments, the linker comprises a monomer, dimer, or polymer of aminoalkanoic acid. In some embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, the linker comprises a monomer, dimer, or polymer of aminohexanoic acid (Ahx). In some embodiments, the linker is based on a carbocyclic moiety (e.g., cyclopentane, cyclohexane). In other embodiments, the linker comprises a polyethylene glycol moiety (PEG). In other embodiments, the linker comprises amino acids. In some embodiments, the linker comprises a peptide. In some embodiments, the linker comprises an aryl or heteroaryl moiety. In some embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated.

In some embodiments, a linker comprises the amino acid sequence of any one of SEQ ID NOs: 10-22 or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 17), and SGGS (SEQ ID NO: 10). In some embodiments, the linker comprises SEQ ID NO: 22.

Fusion Proteins Bound to Lipids (Lipoproteins) and/or Therapeutics

In some embodiments, the fusion proteins as described above, a further modified to comprise a therapeutic. In some embodiments, the therapeutic is a lipid. In some embodiments, the therapeutic interacts with a lipid receptor. In some embodiments, the therapeutic is selected from the group consisting of S1P receptor agonists or antagonists; or prostaglandin agonists or antagonists; or combination thereof.

S1P receptor agonists and antagonists are well known in the art and are described in Park S J et al. Biomolecules & therapeutics 25.1 (2017): 80, which is incorporated by references in its entirety. In some embodiments, S1p receptor antagonists are selected from the group consisting of, MT-1303 and JTE-013. In some embodiments, S1p receptor agonists are selected from the group consisting of SEW2871, KRP-203, Siponimod (BAF312), AUY954, Ponesimod (ACT-128800), Ceralifimod (ONO-4641), GSK2018682, Ozanimod (RPC1063), CS-0777, and Fingolimod (FTY720, Gilenya).

In some embodiments, the therapeutic inhibits production of prostaglandins. In some embodiments, the therapeutic is a non-steroidal anti-inflammatory drug that inhibits prostaglandin production.

Prostaglandin receptor agonists and antagonists are well known in the art and are described in Sharif N A et al. British journal of pharmacology 176.8 (2019): 1059-1078, which is incorporated by reference in its entirety. In some embodiments, prostaglandin receptor agonists are selected from the group consisting of Cloprostenol, Fluprostenol (travoprost acid), 16-Phenoxy-ω-tetranor-PGF2α, 17-Phenyl-ω-trinor-PGF2α (bimatoprost acid), 13,14-Dihydro-17-phenyl-ω-trinor-PGF2α (latanoprost-free acid) (PhXA85), AFP-172 (tafluprost acid), and AL-12182 acid (AL-12180). In some embodiments, prostaglandin receptor antagonists are selected from the group consisting of PGF2α dimethylamide, PGF2α dimethylamine, Phloretin, Glibenclamide, tolbutamide, AL-8810, AL-3138, AS604872, THG-113.31, PDC113.824, AL-8810, and AGN 211377.

In some embodiments, the fusion protein is a lipoprotein bound to a lipid. In some embodiments, the lipid is a therapeutic. In some embodiments, the lipid is selected from the group consisting of prostaglandin, sphingosine 1-phosphate (S1P), a leukotriene. In some embodiments, the prostaglandin is Iloprost. In some embodiments, the prostaglandin is selected from the group consisting of prostaglandin E2 (PGE2), prostacyclin (PGI2), prostaglandin D2 (PGD2), and prostaglandin F2α (PGF2α). In some embodiments, the lipid is S1P. In some embodiments, the lipid binds to the ApoA1 of the fusion protein.

### Nanoparticles

In some embodiments, the lipoprotein as described above is incorporated into a nanoparticle. In some embodiments, the nanoparticle is a liposome. In some embodiments, the nanoparticle is a nanodisc. A nanodisc is a discoidal particle comprising a lipid bilayer and proteins that form disc like structure, where the proteins encircle the lipid bilayer (Denisov, Ilia G., and Stephen G. Sligar. Nature structural & molecular biology 23.6 (2016): 481-486, which is incorporated by reference in its entirety). In some embodiments, the nanoparticle is similar to an HDL-like nanodisc. In some embodiments, the lipoprotein incorporated into a nanoparticle (e.g., a nanodisk) is bound to a phospholipid. In some embodiments, the phospholipid is phosphatidyl choline.

In some embodiments, the nanoparticle comprises fusion protein as described above that has not been lipidated (NL-fusion protein) and fusion protein that has been lipidated (e.g., a lipoprotein as described above). In some embodiments, the nanoparticle comprises at least 10% (e.g., at least 10%, at least 20%, at least 30%, at least 40%, at least 50%, at least 60%, at least 70%, at least 80%, at least 90%, at least 95%, or at least 99%) lipoprotein. In some embodiments, the nanoparticle comprises 10%-20%, 20%-30%, 30%-40%, 40%-50%, 50%-60%, 60%-70%, 70%-80%, 80%-90% or 90%-100% lipoprotein compared to NL-fusion protein. In some embodiments, the nanoparticle comprises 100% lipoprotein. In some embodiments, the nanoparticle comprises a ratio of lipoprotein to NL-fusion protein of 1:10, 1:7, 1:5, 1:3, 1:2, 1:1, 2:1, 3:1, 5:1, 7:1, or 10:1. In some embodiments the nanoparticle comprises 3 lipoproteins for every 7 NL-fusion proteins. In some embodiments, the nanoparticle comprises 30% lipoprotein and 70% NL-fusion protein. In some embodiments, the nanoparticle comprises a fusion protein bound to a therapeutic as described above. In some embodiments, the nanoparticle comprises a therapeutic as described above.

### Modified Fusion Proteins

In some embodiments, the fusion protein described herein comprises a modification. When the fusion protein is referred to herein, it encompasses all its variants and derivatives. Polypeptides comprising modifications have additional features other than amino acid contents. As used herein, a “modification” or “derivative” of a protein or polypeptide (e.g., the fusion protein described herein) produces a modified or derivatized polypeptide, which is a form of a given peptide that is chemically modified relative to the reference peptide, the modification including, but not limited to, oligomerization or polymerization, modifications of amino acid residues or peptide backbone, cross-linking, cyclization, conjugation, PEGylation, glycosylation, acetylation, phosphorylation, acylation, carboxylation, lipidation, thioglycolic acid amidation, alkylation, methylation, polyglycylation, glycosylation, polysialylation, adenylylation, PEGylation, fusion to additional heterologous amino acid sequences, or other modifications that substantially alter the stability, solubility, or other properties of the peptide while substantially retaining the activity of the polypeptides described herein. It is to be understood that the fusion protein comprising such modifications, are cross-linked, cyclized, conjugated, acylated, carboxylated, lipidated, acetylated, thioglycolic acid amidated, alkylated, methylated, polyglycylated, glycosylated, polysialylated, phosphorylated, adenylylated, PEGylated, or combination thereof. In some embodiments, the modified fusion protein of the present disclosure may contain non-amino acid elements, such as polyethylene glycols, lipids, poly- or mono-saccharide, and phosphates. The fusion protein of the present disclosure, may comprise the modifications disclosed herein at the C-terminus (e.g., C-terminal amidation), N-terminus (e.g., N-terminal acetylation). Terminal modifications are useful, and are well known, to reduce susceptibility to proteinase digestion, and therefore serve to prolong half-life of the polypeptides in solutions, particularly biological fluids where proteases may be present. In some embodiments, the fusion proteins described herein are further modified within the sequence, such as, modification by terminal-NH₂ acylation, e.g., acetylation, or thioglycolic acid amidation, by terminal-carboxylamidation, e.g., with ammonia, methylamine, and the like terminal modifications.

Terminal modifications are useful, to reduce susceptibility by proteinase digestion, and therefore can serve to prolong half-life of the polypeptides in solution, particularly in biological fluids where proteases may be present. Amino terminus modifications include methylation (e.g., —NHCH3 or —N(CH3)2), acetylation (e.g., with acetic acid or a halogenated derivative thereof such as a-chloroacetic acid, a-bromoacetic acid, or a-iodoacetic acid), adding a benzyloxycarbonyl (Cbz) group, or blocking the amino terminus with any blocking group containing a carboxylate functionality defined by RCOO— or sulfonyl functionality defined by R—SO2-, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the polypeptide. In certain embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the peptides described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art, for example, in U.S. Patent Application No. 20090035814; Muralidharan and Muir, 2006, Nat Methods, 3:429-38; and Lockless and Muir, 2009, Proc Natl Acad Sci USA. June 18, Epub. C-terminal functional groups of the peptides described herein include amide, amide lower alkyl, amide di(lower alkyl), lower alkoxy, hydroxy, and carboxy, and the lower ester derivatives thereof, and the pharmaceutically acceptable salts thereof.

In some embodiments, the fusion proteins described herein are phosphorylated. One can also readily modify peptides by phosphorylation, and other methods (e.g., as described in Hruby, et al. (1990) Biochem J. 268:249-262). In some embodiments, one can also replace the naturally occurring side chains of the genetically encoded amino acids (or the stereoisomeric D amino acids) with other side chains, for instance with groups such as alkyl, lower (C1-6) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocycles. For example, proline analogues in which the ring size of the proline residue is changed from 5 members to 4, 6, or 7 members can be employed.

Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl groups. These heterocyclic groups can be substituted or unsubstituted. Where a group is substituted, the substituent can be alkyl, alkoxy, halogen, oxygen, or substituted or unsubstituted phenyl.

In some embodiments, the fusion proteins described herein may be attached to one or more polymer moieties. In some embodiments, these polymers are covalently attached to the fusion proteins of the disclosure. In some embodiments, for therapeutic use of the end product preparation, the polymer is pharmaceutically acceptable. One skilled in the art will be able to select the desired polymer based on such considerations as whether the polymer-peptide conjugate will be used therapeutically, and if so, the desired dosage, circulation time, resistance to proteolysis, and other considerations.

Suitable polymers include, for example, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and α,β-Poly[(2-hydroxyethyl)-DL-aspartamide, and the like, or mixtures thereof. Such a polymer may or may not have its own biological activity. The polymers can be covalently or non-covalently conjugated to the fusion protein. Methods of conjugation for increasing serum half-life and for radiotherapy are known in the art, for example, in U.S. Pat. Nos. 5,180,816, 6,423,685, 6,884,780, and 7,022,673, which are hereby incorporated by reference in their entirety.

In some embodiments, the fusion protein described herein may be attached to one or more water soluble polymer moieties. The water soluble polymer may be, for example, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl-pyrrolidone)polyethylene glycol, propropylene glycol homopolymers, polypropylene oxide/ethylene oxide copolymers, and polyoxyethylated polyols. A preferred water soluble polymer is PEG.

The polymer may be of any molecular weight, and may be branched or unbranched. The average molecular weight of the reactant PEG is preferably between about 3,000 and about 50,000 daltons (the term “about” indicating that in preparations of PEG, some molecules will weigh more, and some less, than the stated molecular weight). More preferably, the PEG has a molecular weight of from about 10 kDa to about 40 kDa, and even more preferably, the PEG has a molecular weight from 15 to 30 kDa. Other sizes may be used, depending on the desired therapeutic profile (e.g., duration of sustained release desired; effects, if any, on biological activity; ease in handling; degree or lack of antigenicity; and other effects of PEG on a therapeutic peptide known to one skilled in the art).

The number of polymer molecules attached may vary; for example, one, two, three, or more water-soluble polymers may be attached to a peptide of the disclosure. The multiple attached polymers may be the same or different chemical moieties (e.g., PEGs of different molecular weight).

In certain embodiments, PEG may be attached to at least one terminus (N-terminus or C-terminus) of the fusion protein described herein. In some embodiments, PEG may be attached to a linker moiety of the fusion protein. In some embodiments, the linker contains more than one reactive amine capable of being derivatized with a suitably activated PEG species.

PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity), and increase the hydrodynamic size (size in solution) of the agent which prolongs its circulatory time by reducing renal clearance. PEGylation can also provide water solubility to hydrophobic drugs and proteins. PEGylation, by increasing the molecular weight of a molecule, can impart several significant pharmacological advantages over the unmodified form, such as: improved drug solubility, reduced dosage frequency, without diminished efficacy with potentially reduced toxicity, extended circulating life, increased drug stability, and enhanced protection from proteolytic degradation. In addition, PEGylated drugs are have wider opportunities for new delivery formats and dosing regimens. Methods of PEGylating molecules, proteins and peptides are well known in the art, e.g., as described in U.S. Pat. Nos. 5,766,897; 7,610,156; 7,256,258 and the International Application No. WO/1998/032466.

Encompassed herein are conjugates of the fusion protein herein. The fusion proteins can be conjugated to other polymers in addition to polyethylene glycol (PEG). The polymer may or may not have its own biological activity. Further examples of polymer conjugation include but are not limited to polymers such as polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and α,β-Poly[(2-hydroxyethyl)-DL-aspartamide, and the like, or mixtures thereof.

Conjugation to a polymer can improve serum half-life, among other effects. A variety of chelating agents can be used to conjugate the peptides described herein. These chelating agents include but are not limited to ethylenediaminetetraacetic acid (EDTA), diethylenetriaminopentaacetic acid (DTPA), ethyleneglycol-0,0′-bis(2-aminoethyl)-N,N,N′,N′-tetraacetic acid (EGTA), N,N′-bis(hydroxybenzyl)ethylenediamine-N,N′-diacetic acid (HBED), triethylenetetraminehexaacetic acid (TTHA), 1,4,7,10-tetra-azacyclododecane-N,N′,N″,N′″-tetraacetic acid (DOTA), 1,4,7,10-tetraazacyclotridecane-1,4,7,10-tetraacetic acid (TITRA), 1,4,8,11-tetraazacyclotetradecane-N,N′,N″,N′″-tetraacetic acid (TETA), and 1,4,8,11-tetraazacyclotetradecane (TETRA). Methods of conjugation are well known in the art, for example, P. E. Thorpe, et. al, 1978, Nature 271, 752-755; Harokopakis E., et. al., 1995, Journal of Immunological Methods, 185:31-42; S. F. Atkinson, et. al., 2001, J. Biol. Chem., 276:27930-27935; and U.S. Pat. Nos. 5,601,825, 5,180,816, 6,423,685, 6,706,252, 6,884,780, and 7,022,673, which are hereby incorporated by reference in their entirety.

Other methods for stabilizing peptides known in the art may be used with the methods and compositions described herein. For example, using D-amino acids, using reduced amide bonds for the peptide backbone, and using non-peptide bonds to link the side chains, including, but not limited to, pyrrolinone and sugar mimetics can each provide stabilization. The design and synthesis of sugar scaffold peptide mimetics are described by Hirschmann et al. (J. Med. Chem., 1996, 36, 2441-2448, which is incorporated herein by reference in its entirety). Further, pyrrolinone-based peptide mimetics present the peptide pharmacophore on a stable background that has improved bioavailability characteristics (see, for example, Smith et al., J. Am. Chem. Soc. 2000, 122, 11037-11038), which is incorporated herein by reference in its entirety.

All combinations of the different modifications and derivatizations are envisioned for the fusion protein described herein. Modifications, derivatives and methods of reprivatizing polypeptides are described in Published International Application WO 2010/014616, the contents of which are incorporated herein by reference.

### Methods of Producing Fusion Proteins

Other aspects of the present disclosure provide methods of producing the fusion protein. The fusion protein will generally be produced by expression form recombinant nucleic acids in appropriate cells (e.g., bacterial cell or eukaryotic cells) and isolated. To produce the fusion protein, nucleic acids encoding the fusion protein may be introduced to a cell (e.g., a bacterial cell or a eukaryotic cell such as a yeast cell or an insect cell. The cells may be cultured under conditions that allow the fusion protein to express from the nucleic acids encoding the fusion protein. Fusion proteins comprising a signal peptide can be secreted, e.g., into the culturing media and can subsequently be recovered. The fusion protein may be isolated using any methods of purifying a protein known in the art.

### Nucleic Acids Encoding Fusion Proteins

The nucleic acids encoding the fusion proteins described herein may be obtained, and the nucleotide sequence of the nucleic acids determined, by any method known in the art. Non-limiting, exemplary nucleotide sequence encoding the fusion protein or variants described herein are provided in Table 1, e.g., SEQ ID NO: 25 or 26. In some embodiments, the nucleic acid sequence encoding the A1M fusion protein is at least 70% (e.g. at least 70%, at least 80%, at least 90%, at least 95%, or at least 99%) identical to SEQ ID NO: 25 or 26. In some embodiments, the nucleic acid sequence encoding the A1M fusion protein comprises SEQ ID NO: 25 or 26. In some embodiments, the nucleic acid sequence encoding the A1M fusion protein consists of SEQ ID NO: 25 or 26.

One skilled in the art is able to identify the nucleotide sequence encoding the fusion protein from the amino acid sequence of the fusion protein. The nucleic acids encoding the fusion protein of the present disclosure, may be DNA or RNA, double-stranded or single stranded. In some embodiments, the nucleotide sequence encoding the fusion protein may be codon optimized to adapt to different expression systems (e.g., for mammalian expression).

In some embodiments, the nucleic acid is comprised within a nucleic acid construct. In some embodiments, the construct is a plasmid or a vector such as an expression vector. In some embodiments, the vector is a viral vector. In some embodiments, the viral vector is a viral expression vector. In some embodiments, the vector comprises a promoter operably linked to the nucleic acid.

A variety of promoters can be used for expression of the fusion proteins described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the fusion proteins described herein. In some embodiments, the expression of the fusion proteins described herein is regulated by a constitutive, an inducible or a tissue-specific promoter.

### Cells Comprising a Fusion Protein

The host cells used to express the fusion proteins described herein may be either prokaryotic cells like bacterial cells such as Escherichia coli, or, preferably, eukaryotic cells. In particular, mammalian cells, such as Chinese hamster ovary cells (CHO), in conjunction with a vector such as the major intermediate early gene promoter element from human cytomegalovirus is an effective expression system for immunoglobulins (Foecking et al. (1986) “Powerful And Versatile Enhancer-Promoter Unit For Mammalian Expression Vectors,” Gene 45:101-106; Cockett et al. (1990) “High Level Expression Of Tissue Inhibitor Of Metalloproteinases In Chinese Hamster Ovary Cells Using Glutamine Synthetase Gene Amplification,” Biotechnology 8:662-667).

A variety of host-expression vector systems may be utilized to express the fusion proteins described herein. Such host-expression systems represent vehicles by which the coding sequences of the isolated fusion proteins described herein may be produced and subsequently purified, but also represent cells which may, when transformed or transfected with the appropriate nucleotide coding sequences, express the fusion proteins described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the fusion proteins described herein; yeast (e.g., Saccharomyces pichia) transformed with recombinant yeast expression vectors containing sequences encoding the fusion proteins described herein; insect cell systems infected with recombinant virus expression vectors (e.g., baclovirus) containing the sequences encoding the fusion proteins described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the fusion proteins described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the fusion proteins being expressed. For example, when a large quantity of such a protein is to be produced, for the generation of pharmaceutical compositions of fusion proteins described herein, vectors which direct the expression of high levels of fusion protein products that are readily purified may be desirable. Such vectors include, but are not limited, to the E. coli expression vector pUR278 (Rüther et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The lpp Gene Of Escherichia Coli,” Nucleic Acids Res. 13:3101-3110; Van Heeke et al. (1989) “Expression Of Human Asparagine Synthetase In Escherichia Coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa (Nagai et al. (1985) “Oxygen Binding Properties Of Human Mutant Hemoglobins Synthesized In Escherichia Coli,” Proc. Nat. Acad. Sci. USA 82:7252-7255, and reviewed in Jenny et al. (2003) “A Critical Review Of The Methods For Cleavage Of Fusion Proteins With Thrombin And Factor Xa,” Protein Expr. Purif. 31:1-11, each of which is incorporated by reference herein in its entirety)), enterokinase (Collins-Racie et al. (1995) “Production Of Recombinant Bovine Enterokinase Catalytic Subunit In Escherichia Coli Using The Novel Secretory Fusion Partner DsbA,” Biotechnology 13:982-987 hereby incorporated by reference herein in its entirety)), furin, and AcTEV (Parks et al. (1994) “Release Of Proteins And Peptides From Fusion Proteins Using A Recombinant Plant Virus Proteinase,” Anal. Biochem. 216:413-417 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express fusion proteins described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion proteins described herein. Such engineered cell lines may be particularly useful in screening and evaluation of fusion proteins that interact directly or indirectly with the fusion proteins described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11: 223-232), hypoxanthine-guanine phosphoribosyltransferase (Szybalska et al. (1992) “Use Of The HPRT Gene And The HAT Selection Technique In DNA-Mediated Transformation Of Mammalian Cells First Steps Toward Developing Hybridoma Techniques And Gene Therapy,” Bioessays 14: 495-500), and adenine phosphoribosyltransferase (Lowy et al. (1980) “Isolation Of Transforming DNA: Cloning The Hamster aprt Gene,” Cell 22: 817-823) genes can be employed in tk−, hgprt− or aprt− cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78: 1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78: 2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al. (1981) “A New Dominant Hybrid Selective Marker For Higher Eukaryotic Cells,” J. Mol. Biol. 150:1-14.

The expression levels of the fusion described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a fusion protein described herein is amplifiable, increase in the level of inhibitor present in culture of host cell will increase the number of copies of the marker gene. Since the amplified region is associated with the nucleotide sequence of a fusion protein described herein or a fusion protein described herein, production of the fusion protein will also increase (Crouse et al. (1983) “Expression And Amplification Of Engineered Mouse Dihydrofolate Reductase Minigenes,” Mol. Cell. Biol. 3:257-266).

Once a fusion described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen, and sizing column chromatography), centrifugation, differential solubility, or by any other standard technique for the purification of polypeptides or antibodies.

In some embodiments, to facilitate purification, e.g., by affinity chromatography, the fusion protein described herein further contains a fusion domain. Well known examples of such fusion domains include, without limitation, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HTS6) fusion partners.

### Methods of Treatment

Further provided herein are methods of using the fusion proteins (e.g., in therapeutic applications). For example, in some embodiments, the fusion protein described herein is formulated in a pharmaceutical composition. Methods of treating a disease or disorder using the fusion protein described herein or the pharmaceutical composition comprising such are also provided. In some embodiments, the method using the fusion protein described herein comprises contacting the fusion protein with S1P. Contacting the fusion protein described herein with S1P results in the formation of a complex between the fusion protein and S1P. In some embodiments, such contacting is carried out in a cell.

In some embodiments, a composition comprising the fusion protein (e.g. the fusion protein, fusion protein bound to a therapeutic, lipoprotein or nanoparticle) as described above is used in methods of treating a disease or disorder associated with reduced level of sphingosine-1-phosphate (S1P), the method comprising administering to a subject in need thereof a therapeutically effective amount of the composition comprising the fusion protein. Such fusion protein binds to S1P and activates a S1P receptor, triggering downstream signaling pathway. In some embodiments, the S1P receptor is S1P1. It is demonstrated herein that the fusion protein-S1P complex specifically activates the S1P1 receptor, compared to other types of S1P receptors, e.g., S1P2 or S1P3. In some embodiments, the S1P receptor (e.g., S1P1) is vascular, i.e., found on the surface of an endothelial cell in a blood vessel.

A disease or disorder “associated with reduced level of S1P” refers to an abnormal condition where the level or activity of S1P, or S1P-triggered signaling pathway is reduced in a subject that has the disease or disorder, compared to a healthy subject. In some embodiments, the level or activity of S1P, or S1P-triggered signaling pathway is reduced by at least 20% in a subject that has the disease or disorder, compared to a healthy subject. For example, the level or activity of S1P, or S1P-triggered signaling pathway may be reduced by at least 20%, at least 30%, at least 40%, at least 50%, at least 60%, at least 70%, at least 80%, at least 90%, or 100% in a subject that has the disease or disorder, compared to a healthy subject. In some embodiments, the level or activity of S1P, or S1P-triggered signaling pathway is reduced by 20%, 30%, 40%, 50%, 60%, 70%, 80%, 90%, or 100% in a subject that has the disease or disorder, compared to a healthy subject.

Administering the fusion protein or the composition comprising such fusion protein described herein to a subject having a disease or disorder associated with reduced level of S1P increased S1P signaling, e.g., by activating S1P receptor such as S1P1. In some embodiments, the S1P signaling is increases by at least 20%, in the presence of the fusion protein, compared to without the fusion protein. For example, the S1P signaling may be increased by at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold, at least 1000-fold, or more, in the presence of the fusion protein, compared to without the fusion protein. In some embodiments, the S1P signaling is increased by 20%, 30%, 40%, 50%, 60%, 70%, 80%, 90%, 100%, 2-fold, 5-fold, 10-fold, 50-fold, 100-fold, 1000-fold, or more, in the presence of the fusion protein, compared to without the fusion protein.

In some embodiments, the diseases or disorders associated with reduced level of S1P include, without limitation: infection, sepsis, diabetes, cardiovascular diseases, retinal vascular diseases, peripheral vascular diseases, metabolic syndromes, and respiratory diseases.

In some embodiments, the diseases or disorders associated with reduced level of S1P include, without limitation: primary and/or secondary resistant hypertension, neurogenic hypertension, gestational hypertension, diabetic hypertension, hypertension of chronic kidney disease, cardiac and non-cardiac reperfusion injury, ischemic injury, stroke, pulmonary edema, myocardial infarction, acute coronary syndrome, angina, atherosclerosis, and age-related macular degeneration.

A disease or disorder “associated with reduced level of prostaglandin” refers to an abnormal condition where the level or activity of prostaglandin, or prostaglandin-triggered signaling pathway is reduced in a subject that has the disease or disorder, compared to a healthy subject. In some embodiments, the level or activity of prostaglandin, or prostaglandin-triggered signaling pathway is reduced by at least 20% in a subject that has the disease or disorder, compared to a healthy subject. For example, the level or activity of prostaglandin, or prostaglandin-triggered signaling pathway may be reduced by at least 20%, at least 30%, at least 40%, at least 50%, at least 60%, at least 70%, at least 80%, at least 90%, or 100% in a subject that has the disease or disorder, compared to a healthy subject. In some embodiments, the level or activity of prostaglandin, or prostaglandin-triggered signaling pathway is reduced by 20%, 30%, 40%, 50%, 60%, 70%, 80%, 90%, or 100% in a subject that has the disease or disorder, compared to a healthy subject.

Administering the fusion protein or the composition comprising such fusion protein described herein to a subject having a disease or disorder associated with reduced level of prostaglandin increased prostaglandin signaling, e.g., by activating prostaglandin receptor. In some embodiments, the prostaglandin signaling is increased by at least 20%, in the presence of the fusion protein, compared to without the fusion protein. For example, the prostaglandin signaling may be increased by at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold, at least 1000-fold, or more, in the presence of the fusion protein, compared to without the fusion protein. In some embodiments, the prostaglandin signaling is increased by 20%, 30%, 40%, 50%, 60%, 70%, 80%, 90%, 100%, 2-fold, 5-fold, 10-fold, 50-fold, 100-fold, 1000-fold, or more, in the presence of the fusion protein, compared to without the fusion protein.

In some embodiments, the fusion protein or a composition comprising the fusion protein is used in methods of treating a subject having a disease or disorder associated with vascular endothelial dysfunction. In some embodiments, the subject has poor blood circulation. In some embodiments, the subject has pulmonary hypertension. In some embodiments, the fusion protein or a composition comprising the fusion protein is used in methods of treating a subject having a disease or disorder associated thrombic inflammation.

In some embodiments, the fusion protein or a composition comprising the fusion protein is used to treat TNFalpha-induced NF-kappaB activation. In some embodiments, the fusion protein or a composition comprising the fusion protein is used to treat diseases associated with TNF-alpha-induced NF-kappaB activation including rheumatoid arthritis (RA), inflammatory bowel disease (IBD), multiple sclerosis, atherosclerosis, systemic lupus erythematosus, type I diabetes, chronic obstructive pulmonary disease or asthma (Liu, Ting, et al. Signal transduction and targeted therapy 2.1 (2017): 1-9, which is incorporated by reference in its entirety). In some embodiments, the fusion protein or a composition comprising the fusion protein is used to decrease TNFalpha-induced NF-kappaB activation.

In some embodiments, the fusion protein or a composition comprising the fusion protein is combined with an anti-thrombin agent to treat a disease described herein. In some embodiments, the anti-thrombin agent is Angiopoietin1 or Activated Protein C (APC). In some embodiments, the disease is selected from the group consisting of diabetic nephropathy, lupus patients who get multiple organ involvement (kidney failure, cognitive disorders, and/or lung inflammation), and COVID-19 syndrome induced blood clots. In some embodiments, S1P and Iloprost bound fusion protein nanoparticles inhibited formyl peptide-stimulated oxidative burst. In some embodiments, Iloprost bound A1M inhibits platelet aggregation.

### Pharmaceutical Compositions

A “pharmaceutical composition,” as used herein, refers to the formulation of the fusion protein, composition comprising a fusion protein (e.g. a fusion protein bound to a therapeutic, a lipoprotein or a nanoparticle) described herein in combination with a pharmaceutically acceptable carrier. In some embodiments, the pharmaceutical compositions further comprises an additional therapeutic agent as described above. The pharmaceutical composition can further comprise additional agents (e.g. for specific delivery, increasing half-life, or other therapeutic agents).

The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the fusion protein from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, an fusion protein of the present disclosure in a composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the fusion protein of the disclosure does not absorb are used.

In other embodiments, the fusion protein of the present disclosure are delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

The fusion protein of the present disclosure can be administered as pharmaceutical compositions comprising a therapeutically effective amount of a binding agent and one or more pharmaceutically compatible ingredients.

In some embodiments, the pharmaceutical composition is formulated in accordance with routine procedures as a pharmaceutical composition adapted for intravenous or subcutaneous administration to a subject, e.g., a human being. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as the nanodisc described herein, which is also suitable for parenteral administration. The particles can be of any suitable structure, such as unilamellar or plurilamellar, so long as compositions are contained therein. The fusion proteins of the present disclosure can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757.

The pharmaceutical compositions of the present disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

In some embodiments, the pharmaceutical composition can be provided as a pharmaceutical kit comprising (a) a container containing a fusion protein of the disclosure in lyophilized form and (b) a second container containing a pharmaceutically acceptable diluent (e.g., sterile water) for injection. The pharmaceutically acceptable diluent can be used for reconstitution or dilution of the lyophilized fusion protein of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an fusion protein of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

The terms “treatment” or “to treat” refer to both therapeutic and prophylactic treatments. If the subject is in need of treatment of a conditions (e.g. pathogenic inflammation) then “treating the condition” refers to ameliorating, reducing or eliminating one or more symptoms associated with the disease or preventing any further progression of the disease. If the subject in need of treatment is one who is at risk of a disease (e.g. pathogenic inflammation), then treating the subject refers to reducing the risk of the subject having the disease or preventing the subject from developing the disease.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof. A subject in need thereof can be a subject who has a risk of developing a disease or disorder associated with reduced S1P, or a subject who has such a disease or disordered.

Pharmaceutically compositions that may be used in accordance with the present disclosure may be directly administered to the subject or may be administered to a subject in need thereof in a therapeutically effective amount. The term “therapeutically effective amount” refers to the amount necessary or sufficient to realize a desired biologic effect. For example, a therapeutically effective amount of a cancer-target liposome associated with the present disclosure may be that amount sufficient to ameliorate one or more symptoms of the disease or disorder. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular pharmaceutically compositions being administered the size of the subject, or the severity of the disease or condition. One of ordinary skill in the art can empirically determine the effective amount of a particular therapeutic compound associated with the present disclosure without necessitating undue experimentation.

Subject doses of the fusion protein described herein for delivery typically range from about 0.1 μg to 10 mg per administration, which depending on the application could be given daily, weekly, or monthly and any other amount of time there between. In some embodiments a single dose is administered during the critical consolidation or reconsolidation period. The doses for these purposes may range from about 10 μg to 5 mg per administration, and most typically from about 100 μg to 1 mg, with 2-4 administrations being spaced, for example, days or weeks apart, or more. In some embodiments, however, parenteral doses for these purposes may be used in a range of 5 to 10,000 times higher than the typical doses described above.

In some embodiments, a fusion protein of the present disclosure is administered at a dosage of between about 1 and 10 mg/kg of body weight of the mammal. In other embodiments, a fusion protein of the present disclosure is administered at a dosage of between about 0.001 and 1 mg/kg of body weight of the mammal. In yet other embodiments, a fusion protein of the present disclosure is administered at a dosage of between about 10-100 ng/kg, 100-500 ng/kg, 500 ng/kg-1 mg/kg, or 1-5 mg/kg of body weight of the mammal, or any individual dosage therein.

The formulations of the present disclosure are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the fusion protein of the present disclosure can be administered to a subject by any mode that delivers the fusion protein to the desired location, e.g., mucosal, injection, systemic, etc. Administering the pharmaceutical composition of the present disclosure may be accomplished by any means known to the skilled artisan. In some embodiments, the fusion protein is administered subcutaneously, intracutaneously, intravenously, intramuscularly, intraarticularly, intraarterially, intrasynovially, intrasternally, intrathecally, intralesionally, or intracranially.

For oral administration, the fusion protein of the present disclosure can be formulated readily by combining the active compound(s) with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, NY, pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The fusion protein can be included in the formulation as fine multi particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a lactose, anhydrous lactose, cellulose, sucrose, modified dextrans and starch. Certain inorganic salts may be also used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The pharmaceutical compositions of the present disclosure, when desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the fusion protein may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The pharmaceutical compositions of the present disclosure and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1

### Abstract

Uncontrolled inflammation, the driver of morbidity and mortality in diseases, occurs due to the imbalance of pro- and anti-inflammatory factors. One such factor is high-density lipoprotein (HDL) particles, which suppress inflammation by acting on vascular endothelial cells (EC), platelets and innate immune cells. Anti-inflammatory activity of HDL is mediated in part by bioactive lipids such as sphingosine 1-phosphate (S1P) and prostacyclin (PGI₂) that act on respective G protein-coupled receptors (GPCR) of target cells. A defined HDL-like particle that chaperones S1P and the stable PGI₂ analog (Iloprost) to suppress thromboinflammatory phenotypes of EC, platelets, and neutrophils was produced. ApoA1-ApoM (A1M) fusion protein forms HDL-like particles, associates stably with S1P, activates S1P receptors and stimulated endothelial barrier formation. A1M also attenuated tumor necrosis factor (TNF-α)-induced NF-κB activation and the expression of leukocyte adhesion protein ICAM-1. The ApoA1 moiety of A1M also bound to Iloprost, a stable PGI₂ analog that enhanced endothelial barrier, either alone or additively with S1P-bound A1M. In neutrophils, S1P and Iloprost bound A1M particles inhibited formyl peptide-stimulated oxidative burst, whilst Iloprost bound A1M completely inhibited platelet aggregation. In a mouse model of sterile inflammation, S1P- and Iloprost-bound A1M suppressed inflammatory responses. Enhancement of endogenous anti-inflammatory mechanisms by A1M-bound mediators may be a novel therapeutic approach to control pathologic inflammation or induce resolution mechanisms.

### Introduction

Vascular endothelial dysfunction is an early characteristic of many acute and chronic diseases. Endothelial dysfunction induced either by infection (viral, bacterial), metabolic stress (diabetes, hypercholesterolemia) or aging, promotes leukocyte extravasation, which leads to parenchymal inflammation and organ dysfunction (Cines et al., 1998; Esper et al., 2006; Pober and Sessa, 2007; Castro et al., 2021). While inhibition of the innate immune response in early phases of acute tissue injury diminishes subsequent tissue/organ damage, enhancement of endothelial cell function is also thought to suppress the magnitude of acute inflammation by inhibiting excessive vascular leak, thrombosis and fibrotic changes (Kiseleva et al., 2018).

Extensive research has delineated multiple functions of plasma high-density lipoproteins (HDL) particles. The major structural protein of HDL is apolipoprotein A1 (ApoA1), an amphipathic polypeptide synthesized by the liver that is subsequently lipidated by ABCA1 and G1 transporters to form disc-like structures with a lipid core (reviewed in Yvan-Charet et al., 2011). The nascent HDL particles remove cholesterol from multiple cell types (Reviewed in Ouimet et al., 2019). Further modifications of HDL include associations with enzymes (cholesterol ester transfer protein), anti-oxidant factors (peroxinase-1) and other regulatory proteins (ApoE). In fact, the HDL proteome is complex and contributes to its heterogeneity (reviewed in Heineke, 2010). Although reverse cholesterol efflux function of HDL has been studied extensively, anti-inflammatory functions have also been described. ApoA1/HDL treatment decreases cytokine and endotoxin-induced NFκB activation in monocytic cells (macrophages and neutrophils) and endothelium (Suzuki et al., 2010). Recent studies suggested that ApoA1 depletion of cholesterol from cell membranes changes the cytokine and toll-like receptor-based signal transduction, thus dampening the inflammatory signaling pathways (Fotakis et al., 2019). An important function of HDL is to protect the vasculature. HDL enhances endothelial-derived nitric oxide secretion, which enhances blood flow, endothelial barrier function attenuating vascular leak and promoting endothelial survival during vascular injury. In addition, HDL suppresses thrombotic and hemostatic mechanisms by enhancing the activity of prostacyclin (PGI₂) and the attenuation of tissue factor expression (Mineo et al 2006).

Apolipoprotein M (ApoM), a member of the lipocalin family of transporter proteins, is anchored by an uncleaved signal peptide to the lipid core in a subpopulation (˜5%) of HDL particles (Christofferson et al, 2006). It was shown that ApoM is the physiological carrier for the bioactive lipid mediator sphingosine 1-phosphate (S1P) in plasma (Christofferson et al, 2011). S1P is a high affinity ligand for five G-Protein coupled receptors (GPCRs) that are known as S1PR1-S1PR5. The S1P/S1PR1 axis is critical for vascular development and the maintenance of vascular barrier function in vitro and in vivo (Proia and Hla, 2015). S1P-bound to HDL mediates unique signaling properties such as attenuation of cytokine-induced NFκB activation and adhesion molecule expression, which prompted us to propose that ApoM-containing HDL acts as a biased agonist for S1PR1 (Galvani et al, 2015). This signaling axis was shown to suppress lymphopoiesis in the bone marrow (Blaho et al, 2015), attenuate endothelial injury in the lung (Ding et al, 2020) and suppress liver fibrosis after injury and promote regeneration (Ding et al, 2016). Recent work suggested that S1PR1 signaling may enable trans-endothelial traverse of HDL particles into the tissue parenchymal spaces (Velagapudi et al, 2021).

Recently, the development of an ApoM-Fc fusion protein that enhances endothelial barrier function, suppresses ischemia-reperfusion injury of the heart and the brain and immune complex-mediated and acid-induced lung injury was shown (Swendeman et al, 2017; Burg et al, 2018, Ding et al, 2020). However, not all the endothelial protective effects of HDL-S1P are mimicked by ApoM-Fc-S1P in vitro and in vivo. The design and characterization of the ApoA1-ApoM fusion protein, which forms nanodiscs, chaperones multiple bioactive lipids (PGI₂ and S1P), protects the endothelium, suppresses neutrophil-mediated vascular injury, and platelet aggregation is described below.

### Materials and Methods

### Creation of the ApoA1-ApoM Fusion

The ApoA1-ApoM fusion was constructed as follows. Plasmids for murine ApoA1 (Cat #MR203500) and murine ApoM (Cat #MR201811) were obtained from OriGene. The cDNAs for each sequence were amplified to incorporate the following properties:

ApoA1: The endogenous Kozak sequence of ApoA1 and the ApoA1 ORF. The stop codon is replaced with a codon for glycine.
ApoM: A linker sequence coding for one copy of the following five amino acid sequence linker (GGGGS (SEQ ID NO: 22) was added to the 5′-end of ApoM by sequential PCR. The signal peptide of ApoM (AA1-20) (SEQ ID NO: 8) was removed and the stop codon was replaced by a glycine followed by a coding sequence to incorporate a 6× Histidine sequence, to facilitate protein purification, followed by a stop codon.
The two PCR products were linked after NOT1 digestion and the final fusion PCR product was cloned into the pCDH-puro (InvitroGen) expression vector.
Primers Used for Cloning were:

ApoA1
For:
(SEQ ID NO: 29)
5′- TTTTCTAGAGAGGAGATCTGCCGCCGCGATCG-3′
REV:
(SEQ ID NO: 30)
5′-TTTGCGGCCGCGTACGTCTGGGCAGTCAGAG-3′
ApoM
For A:
(SEQ ID NO: 31)
5′-GGTGGAGGTGGATCTAATCAGTGCCCTGAGCACAGT-3′
Rev A:
(SEQ ID NO: 32)
5′GATGGTGATGTCCCTTGCTGGACAGCGGGCAGG-3′
For B:
(SEQ ID NO: 33)
5′-TTTGCGGCCGCTTGGTGGAGGTGGATCTAATCAGTG-3′
Rev B (HIS-TAG)
(SEQ ID NO: 34)
5′-TTTGGATCCTCAGTGATGGTGATGGTGATGTCCCTTGCTGG-3′

### Expression and Purification of the ApoA1-ApoM Fusion Protein

The resulting pApoA1-ApoM plasmid was transfected into adherent CHO-S cells (InvitroGen) using PEI (Polyethylenimine; 1 mM stock solution in water; Sigma). Positive transfectants were obtained by selection in Puromycin (30 μg/ml GIBCO) for 4 days. Cells were tested for expression of fusion protein by western blot analysis for ApoA1 (Abcam), ApoM (Abcam) and His-tag (Santa Cruz) expression. The drug-selected recombinant CHO-S cells were adapted to serum-free suspension culture using CHO-S medium (CD Forti CHO; ThermoFisher). For large-scale cultures, cells were seeded at 3-5×10⁵ cells per ml, and maintained in culture to 2-4×10⁶ cells/ml. Cells were removed from culture by centrifugation at 800×g for 10 minutes at 4° C. Cell culture supernatant was further clarified by ultracentrifugation at >100,000×g for 30 minutes at 4° C. The resulting supernatant was incubated with Ni-Sepharose beads (HisPur Ni-NTA resin, Thermofisher) at a final concentration of 2 ml of beads (packed)/500 ml of culture. The slurry was incubated overnight at 4° C. and then beads were concentrated by centrifugation at 10,000×g for 5 minutes at 4° C. Beads were washed with 50 volumes of His-wash buffer (20 mM Tris ph 8, 400 mM NaCl, 10 mM Imidizole) until the flow through did not contain detectable protein. Protein was eluted from the column in His-Wash buffer supplemented with 300 mM Imidizole. The resulting protein fractions were assayed using Bio-Rad protein reagent and were concentrated with Amicon C15 filters. The final purified protein preparation was analyzed by SDS-PAGE and stained with Coomassie Blue (Bio-Rad; 0.5% suspended in 40% Methanol, 10% Acetic acid.)

Preparation of S1P, Loading S1P onto ApoA1-ApoM and Mass Spectrometry Analysis:

### Preparation of S1P:

1 mg of S1P (Avanti Polar lipids) is resuspended in 13.4 ml of Methanol and maintained at 37° C. for 12 hours to achieve complete suspension. The solution is dispersed into 134 μl aliquots and dried under vacuum for 45 minutes to 1 hour at 37° C. Dried S1P is maintained at −20° C. until use.

Loading S1P onto ApoM:

A method for loading S1P onto recombinant ApoM was previously described (Swendeman et al, 2017). Essentially, purified protein was suspended in PBS at 1 mg/ml (˜20 μM) and mixed with 160 μM S1P by gentle pipetting. The sample was subjected to 3 rounds of 30 second sonication in a bath sonicator and allowed to incubate for >24 hours by nutation. The resulting product was subjected to FPLC to separate the free S1P from the ApoA1-ApoM-S1P protein complex and the resulting protein fractions were reconcentrated using Amicon C15 filters.

Loading of S1P onto Albumin

S1P was loaded onto Albumin as described (Lepley et al, 2005). Essentially, a 0.4% solution of PBS-Albumin (Fatty acid free, Sigma) was added to dried S1P and subjected to bath sonication for 3× cycles for 1 minute/cycle and maintained at 4° C. for 24 hours.

### Mass Spectroscopy Analysis of S1P

Mass spectroscopy for S1P was performed as described previously (Engelbrecht et al, 200). The purified S1P bound ApoM or ApoA1-ApoM proteins or plasma from mice injected with these proteins were subjected to S1P Mass spectroscopy analysis using the following protocol. Protein-S1P complexes in PBS were extracted in Methanol (20:1; Methanol:analyte, (Vol/vol).

Loading of Iloprost onto ApoA1-ApoM Chaperone

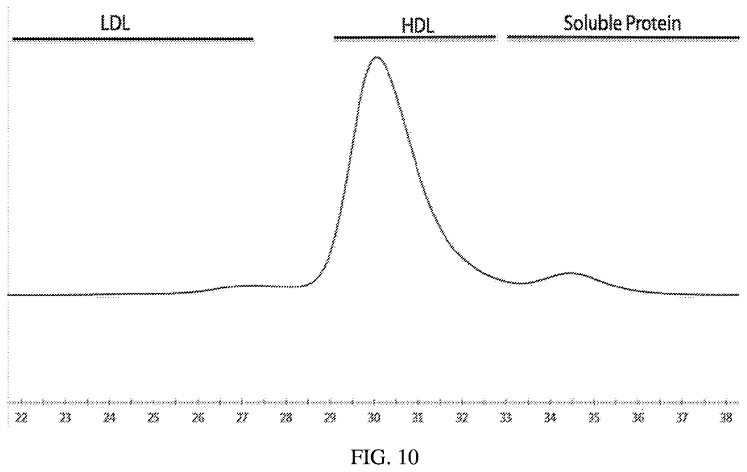

A standard sonication and thermal cycling method for creating lipidated ApoA1-ApoM as described by Schwendeman et al. 2015 or sonication dependent lipidation of ApoA1 as described by Oda et al, 2006 was employed. Either Phosphorylcholine 2 mM (Avanti) or a combination of DMPC and DMPG (7:3 molar ratio) (Avanti/Sigma) were resuspended in Chloroform:Methanol (95:5) and distributed into Eppendorf tubes and dried under vacuum. Lipid was resuspended in PBS buffer, heated at 37° C. for 5-10 minutes and sonicated (20 seconds) to create a lipid suspension. Purified human ApoA1 (Sigma) or purified ApoA1-ApoM protein was resuspended in PBS (10:1 mol/mol) was added to the lipid slurry and subjected to either continuous bath sonication until the suspension exhibited clarification (Indicating lipidation) or subjected to repeated cycles (3-5) of 37° C. heating (5 minutes) followed by 5 minutes of bath Sonication at RT. In each method, Iloprost (Cayman Chemical) was added to the solution at a final concentration of 1 mM. The resulting lipidated solution was subjected to FPLC purification (Swendeman et al, 2017) (FIG. 10) and relevant fractions corresponding to recombinant HDL nanoparticles were collected and concentrated on Amicon Filters.

### S1P and Iloprost TEER Analysis of HUVEC

Transendothelial Electrical Resistance (TEER) was performed on HUVEC as described previously (Swendeman et al, 2017). Essentially, HUVECs were maintained in HUVEC Growth Medium (HGM; M199 medium supplemented with 10% FCS (Corning), 1:100 Pen-Strep (Sigma), 8 mM Glutamine (Sigma), Heparin (Sigma, 100 U, LMW) and Sheep Brain Extract prepared (REF). All cultures were maintained on Fibrobronectin (2 μg/ml) coated plates. For TEER analysis a 96 well ECIS system (96W10idf PET array, Applied BioPhysics) was employed. Wells were coated with Fibronectin (2 μg/ml) resuspended in saline for 30 minutes at room temp. HUVECs were harvested, resuspended in HGM at a cell density of 25-30×10³ cells/well and allowed to adhere overnight. Prior to analysis, culture media was removed and replaced with M199 media supplemented with Pen-Strep/Glutamine and 1% FCS for 30 minutes. For stimulation studies, either Iloprost (Cayman Biochemicals) or Iloprost loaded onto ApoA1 or ApoA1-ApoM chaperones, or S1P prepared on ApoA1-ApoM or ApoM-Fc chaperones were added to cultures and TEER studies were performed from 3-24 hours.

### S1P, Angiopoietin1 (Ang1) and Activated Protein C (APC) TEER Analysis of HUVEC in Response to Thrombin

TEER analysis was performed as described above. Angiopoietin1 (R&D systems) was used at a final concentration of 300 ng/ml for all studies. Thrombin (Millipore Sigma) was used at a final concentration of 1 U/ml for all studies. Activated Protein C was obtained from Enzyme Research laboratories. For initial studies, Ang1 was evaluated alone and in combination with ApoM-Fc-S1P. For Thrombin analysis, Ang1, ApoM-Fc-S1P, and Thrombin were co-added at the initiation of the study. For APC analysis, APC alone or in combination with A1M-S1P was added to the culture for 1 hour prior to the addition of Thrombin and then TEER analysis was performed for an additional 2-8 hours. All data from TEER studies were statistically analyzed and Area under the Curve was determined using GraphPad Prism 7 (GraphPad Software, San Diego, CA).

### Nanobit Analysis of S1P Receptor Activation

The Nano-bit system, which employs split luciferase interactions of GPCRs with betaarrestin or specific Ga/bg complexes in response to GPCR activation (Inoue et al, 2019; Hisano et al, 2019) was used to characterize ApoA1-ApoM nanoparticles containing S1P. Briefly, HEK293A cells maintained in DMEM (GIBCO) supplemented with 10% FCS and Pen-Strep, were dispersed into 6 well plates and allowed to adhere overnight. For functional studies, cells were transfected with appropriate combinations of reporter plasmids. S1PR1-Small bit luciferase and Beta-arrestin-large bit luciferase fusion proteins or Gαᵢ-small bit luciferase combined with Gβ1 and Gγ1-Large bit luciferase were employed as described previously (Hisano et al, 2019). After 24 hrs, cells were harvested, resuspended with the luciferase substrate Coelenterazine (Caymen Chem; 50 μM), dispersed into white opaque-bottom 96-well plates (Greiner) and maintained at RT for 2 hours to quench background. S1P containing samples were added by multi-channel pipetting and the plate was immediately analyzed for fluorescence at 470 nm for 30 minutes in a SpectraMax L 96-well plate reader (Molecular Devices). Data was integrated as described (Hisano et al, 2019).

### Inhibition of TNF-α Dependent NF-κB Activation

TNFα-dependent NF-κB signaling was determined by using an NFκB-Luciferase based reporter assay system (pGL4.32[luc2P/NF-κB-RE/Hygro]; Promega). A stable NFκB reporter cell line in human microvascular endothelial cells (HMEC-1; ATCC) was created. HMEC were maintained in 10% FCS/Pen-Strep MCDB media supplemented with 2 mM L-Glutamine (Sigma), EGF (2 ng/ml; R&D systems) and Hydrocortisone (1 ng/ml; Sigma). Using TNFα (10 ng/ml; R&D Systems), both dose and temporal analyses were performed to establish optimal induction of the Luciferase response (Data not shown). The 5×10⁵ reporter cells were distributed to 12 well plates and allowed to adhere for 24 hours. Media was replaced with MCDB media supplemented with 1% FCS, L-Glutamine. Cells were pre-treated with either media, ApoA1 (Purified Human ApoA1; Sigma), ApoA1-ApoM, ApoA1-ApoM-S1P, ApoM-Fc-S1P, or Albumin-S1P for 10 minutes and 2 ng/ml of TNFα. Samples were extracted using cell lysis buffers (Promega), luciferin substrate was added and the plates were measured for fluorescence at 470 nm for 8-15 minutes in a SpectraMax L 96-well plate reader (Molecular Devices).

### Inhibition of TNF-α-Induced ICAM-1 Expression in HUVEC

HUVECs were plated as described above. After 24 hours cells were shifted to M199 media supplemented with 1% FCS, 8 mM Glutamine, 1× Pen-Strep. After 30 minutes, cells were pretreated for 10 minutes with media, ApoM-Fc-S1P (100 nM S1P), Iloprost (100 nM), ApoA1-ApoM (200 μg/ml), ApoA1-ApoM-Iloprost (200 ug/ml; 100 nM Iloprost) or ApoA1-ApoM-S1P (200 μg/ml 100 nM S1P) or in combinations and subsequently treated with TNF-α (10 ng/ml). After 5 hours, cells were lysed with cell lysis buffer (TBS-T; 20 mM Tris-pH8, 160 mM NaCl, 1% Triton, 1× protease inhibitor cocktail (Sigma)), collected and centrifuged at 20,000×g for 5 minutes at 4° C., extracts were analyzed by 10% SDS-PAGE, transferred to nitrocellulose membrane (Bio-Rad), blocked in 5% milk, and probed with antibodies to ICAM-1 (1:1000, sc-8439, Santa Cruz Biotech) and Actin (1:5000, sc-8432, Santa Cruz Biotech). Blots were developed with appropriate secondary antibodies linked to HRP and visualized by chemiluminescence (Immobilon Western, EMD Millipore) using a ChemiDoc Imaging (Biorad).

### Iloprost-Induced Activation of the CREB-Luciferase Signaling

In order to evaluate the activity of lipoprotein-bound Iloprost to activate the IP GPCR, the downstream cAMP-dependent CREB-luciferase reporter system (pGL4.29 [luc2P/CRE/Hygro]; Promega) was examined. HEK293T or A cells were maintained in DMEM (InVitrogen) supplemented with 10% FCS and Pen-Strep (Corning). Cells were harvested and dispersed 2-3×10⁵ cells/well in 6 well plates and allowed to adhere for 24 hours. Media was replaced and cells were transfected using PEI (Polyethylenimine; 1 mM stock solution in water; Sigma) with either 0.3 μg/well of reporter plasmid alone or co-transfected with pCMV-PI, which encoded the IP receptor. After 24 hours, media was replaced and cells were incubated for 8 hours at 37° C. with media containing either vehicle, Iloprost (5 nM-100 nM), or ApoA1-ApoM-Iloprost (1-32 μg/ml) by titration. Samples were lysed, luciferin was added and the plates were quantified for fluorescence at 470 nm for 30 minutes in a SpectraMax L 96-well plate reader (Molecular Devices). Data was integrated as described. Untransfected HEK293T or A cells did not respond to Iloprost stimulation, and gained responsiveness upon IP GPCR expression.

### Inhibition of Human Platelet Aggregation In Vitro

Whole blood samples were drawn from healthy donors in the presence of 10% sodium citrate (Sigma, 5577-50 ml) and were spun at 1000 rpm using Beckman centrifuge (GS-6K) for 20 min. The top Platelet-rich Plasma (PRP) layer was collected resting at a 37° C. water bath for 30 minutes. PRP was diluted with the one-fifth volume of the acid-citrate-dextrose buffer, Prostaglandin E₁ (PGE₁) was added to a final concentration of 0.15 μM. The mixture was spun in a conical-bottom tube at 2000 g for 10 min, and the platelet pellet was collected and suspended in pre-warmed HEPES-Tyrode-Glucose Buffer (HTG). The platelet concentration was determined and adjusted to ˜2.5×10⁵/μL with HTG on a Hematology cell counter System (Drew Scientific, 850FS).

Light Transmission Aggregometry was used to evaluate the platelet response to agonists and antagonists. In a 4-channel aggregometer (Platelet ionized calcium aggregometer, Chronolog Corp Model 660), human platelets were stirred in a cuvette at 37° C. Human platelets were incubated with A1M-Iloprost, ApoA1-Iloprost or A1M-S1P alone or in combination for ˜10 min before adding thrombin receptor (PAR-1) agonist SFLLRN peptide (2 μM). Data were analyzed using AGGRO/LINK software package Ver 5.2.5 and Microsoft office professional plus 2013.

### Inhibition of Neutrophil Reactive Oxygen Species (ROS) Release

A1M, A1M-Iloprost and A1M-S1P were assayed for inhibition of f-Met-Leu-Phe (f-MLP) dependent Reactive Oxygen Species generation in isolated Neutrophils as follows. Peritoneal Neutrophils were isolated from C57Bl/6 mice after thioglycolate elicitation. Thioglycolate (2 ml of 2% v/v) resuspended in water was administered by intraperitoneal injection. After 4 hours, mice were euthanized and 5 ml of HBSS was injected intraperitoneally, the abdomen was messaged for 1 minute and the peritoneal fluid was removed. The resulting cells were pelleted, RBCs were lysed with ACK buffer, re-pelleted and resuspended in PBS-glucose. Cells were counted, 5×10⁵ cells were dispersed into 96 well clear bottom plates containing luminol 100 mM and 10 U/ml of horseradish peroxidase and read for blank background. F-Met-Leu-Phe (fMLP; 10 μM) was added and plates were read on a SpectraMaxL1 (Molecular Devices, San Jose, CA) over 5 minutes as described previously. The area under the curve was calculated by using GraphPad Prism 7 (GraphPad Software, San Diego, CA).

### Animals

C57Bl/6 mice were obtained from Jackson Labs. All in vivo experiments were performed according to approved experimental protocols by IACUC at Boston Children's Hospital.

### Results

### Production of ApoA1-ApoM-S1P (A1M-S1P) Nanodiscs

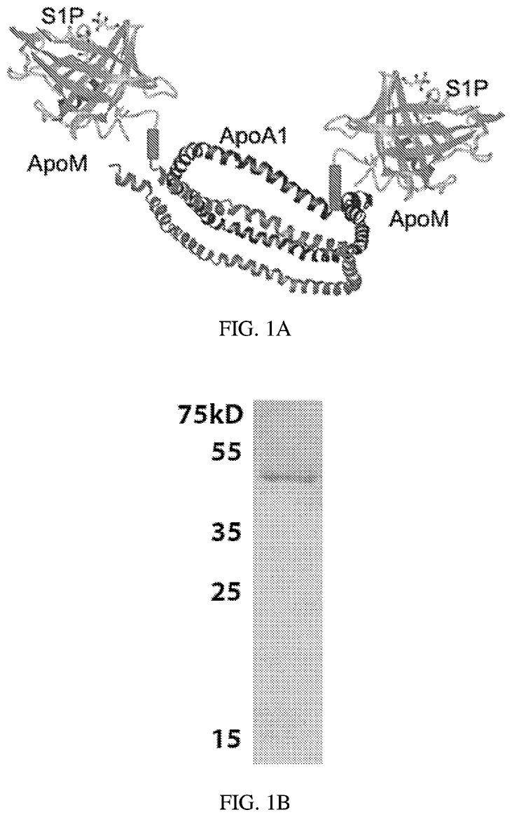

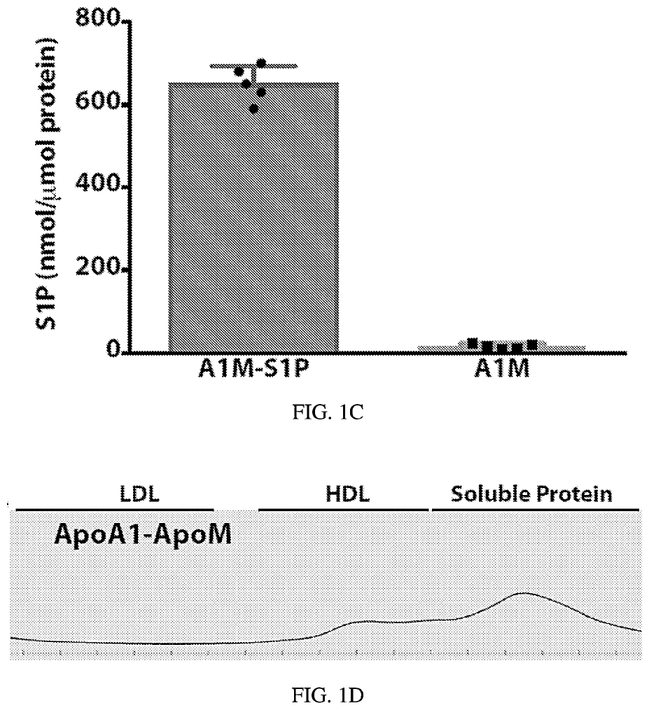

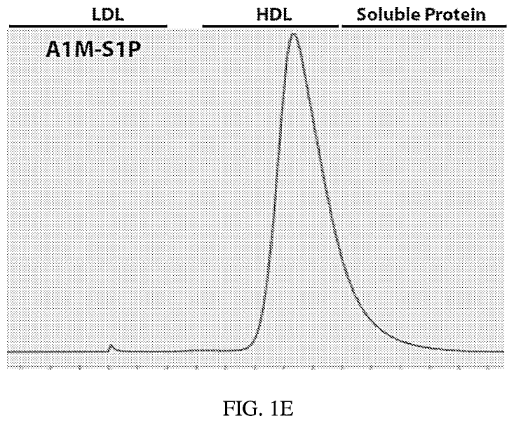

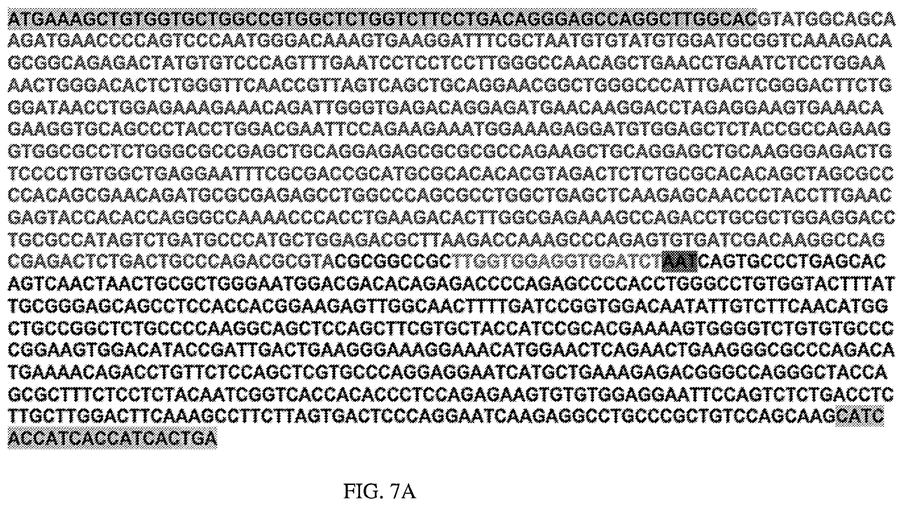

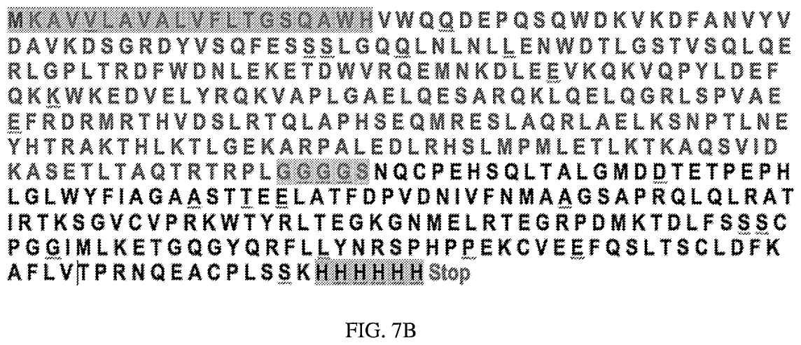

An Fc-fusion with the S1P chaperone protein ApoM (ApoM-Fc) was created and demonstrated that it enhances endothelial cell function in vitro and vascular function in vivo (Swendeman et al., 2017; Burg et al., 2018, Ding et al., 2020). Since the core HDL structural protein ApoA1 also was shown to regulate nitric oxide release in endothelial cells and suppress inflammatory response of myeloid cells (Fotakis et al., 2019, Yuhanna et al, 2001, Mineo et al., 2006), it was reasoned that a fusion protein of ApoA1 and ApoM might exhibit the distinct properties of each molecule. The fusion of ApoA1-ApoM (A1M) fusion protein was constructed with a flexible linker domain (GGGGS (SEQ ID NO: 22)) (FIGS. 7A-7B) (Trinh et al, 2004). In addition, a 6×-Histidine tag was added on the carboxyl-terminus to enable ease of protein purification (FIGS. 7A-7B). A1M protein from conditioned media collected from stably-transfected CHO-S cells was expressed and purified. As shown in FIG. 1B, a homogenous preparation of purified A1M protein as obtained.

Sonication of A1M and concentrated S1P solution and prolonged incubation was done to load S1P to A1M. Subsequently, A1M-S1P was separated from the free S1P by FPLC gel filtration chromatography. Under these conditions, ˜54-68 mol % of A1M protein was loaded with S1P assuming a 1:1 stoichiometry (N=5; FIG. 1C). In contrast, unloaded purified A1M was <0.5 mol % loaded with S1P, presumably from endogenous sources (CHO cells or FBS-containing cell culture medium).

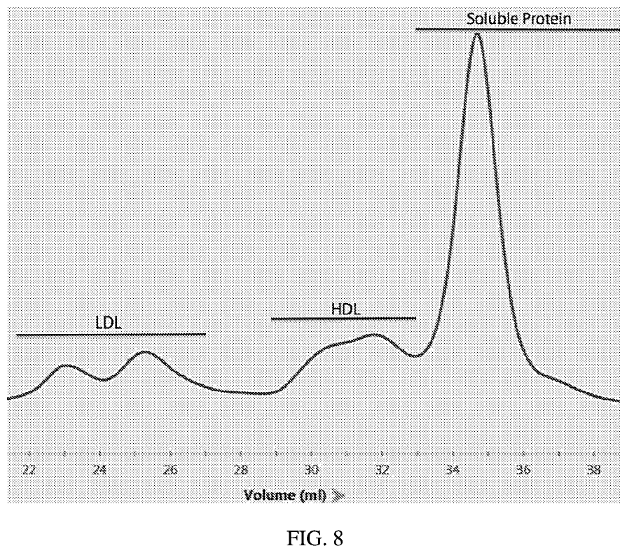

Since the ApoA1 moiety of HDL can be lipidated in vitro to form nanodiscs of reconstituted HDL which could accommodate bioactive lipid mediators such as S1P, A1M was lipidated with mixture of phosphatidyl choline and S1P as described (Schwendeman et al., 2015). FPLC elution profiles of samples were compared to a reference of fractionated mouse plasma (FIG. 8). Analysis of native A1M reveals a mixture of lipidated protein (˜30%) and unlipidated protein (70%)(FIG. 1D). After the lipidation procedure, the A1M loaded with S1P (FIG. 1E) eluted between HDL and the soluble protein fractions in the FPLC, suggesting efficient conversion to nanodisc configuration.

ApoA1-ApoM-S1P Induces Both b-Arrestin and Gai-Dependent Coupling of the S1P Receptors

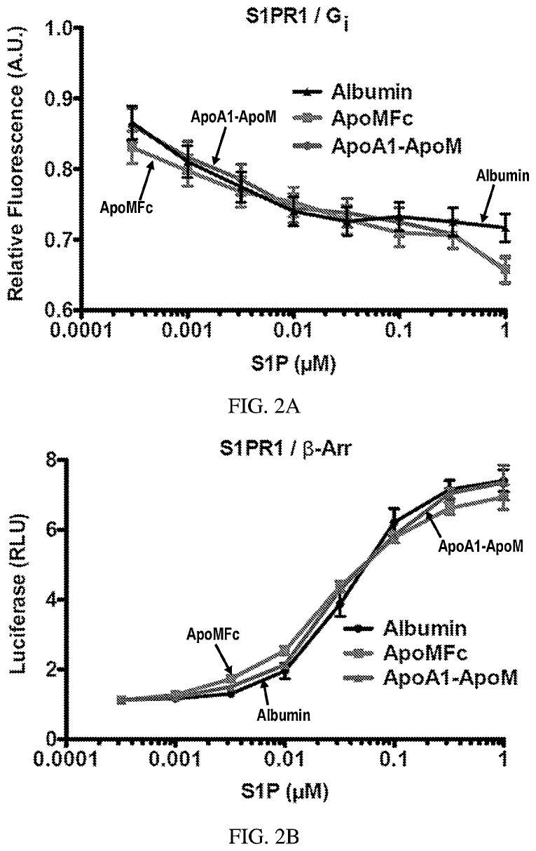

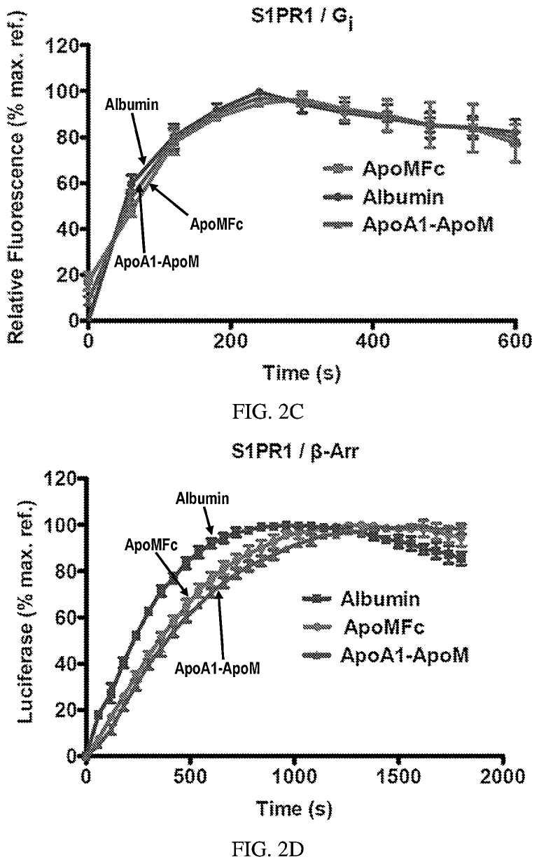

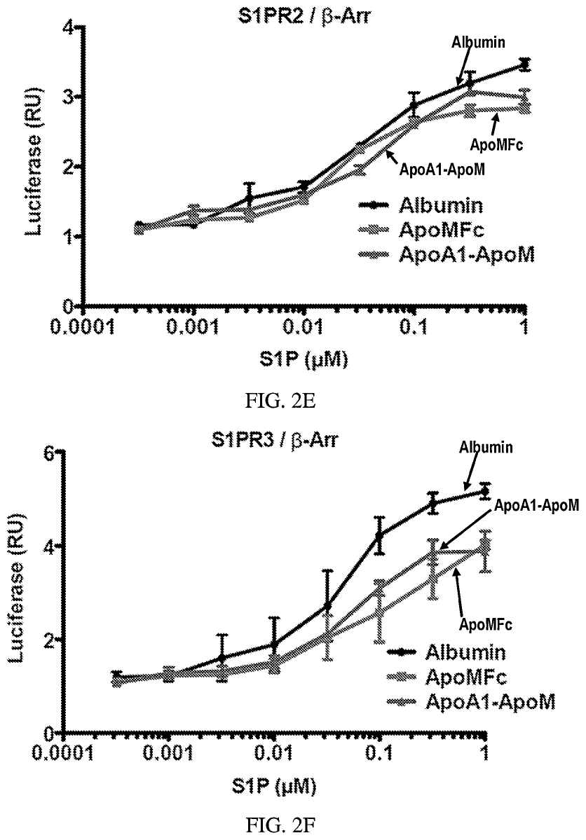

The Nanobit system was used to determine signaling properties of the A1M-S1P nanodiscs activation of S1P receptors (Inoue et al, 2019). In addition, signaling properties of A1M-S1P were compared with albumin-S1P and ApoM-Fc-S1P. Over a 4-log range of ligand stimulation, similar S1PR1-dependent activation of Gai (FIG. 2A) and b-arrestin receptor-coupling (FIG. 2B) was observed for all three chaperones complexed with S1P, suggesting that ApoM chaperone function is not impaired by fusion to either Fc domain or ApoA1. However, temporal analysis of receptor stimulation at saturating ligand concentrations (1 μM S1P) revealed that regardless of chaperone, S1P activated S1PR1/GaI with similar kinetics.

S1P chaperone-dependent β-arrestin activation via S1PR2 and S1PR3 (FIGS. 2E-2F) was also analyzed. Greater maximum stimulation of these receptors with Albumin-S1P was observed, when compared to either ApoM-Fc-S1P or ApoA1-ApoM-S1P, suggesting the signaling from S1PR2 and S1PR3 is greater when S1P is bound to albumin. In contrast, ApoM-bound S1P activates S1PR1 better regardless of association with soluble or nanodisc chaperone.

### ApoA1-ApoM-S1P Specifically Enhances Endothelial Barrier Function and Provides Protection Against Thrombin-Induced Barrier Degradation

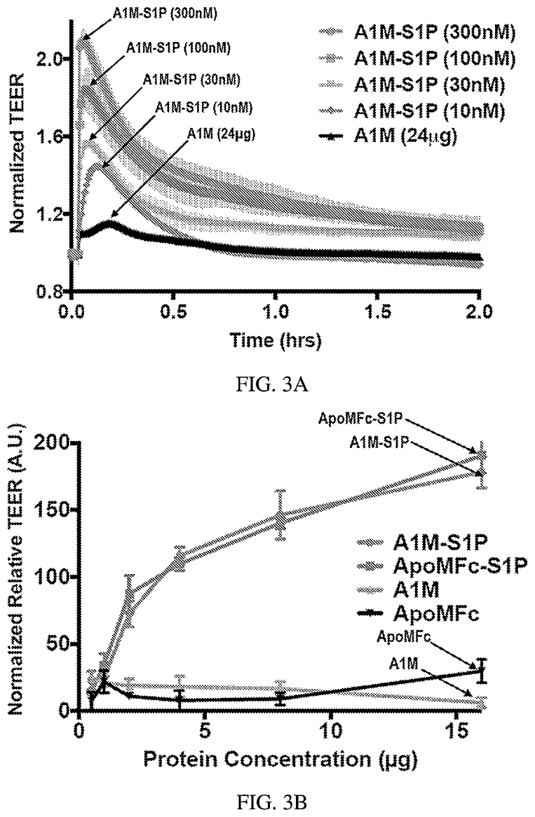

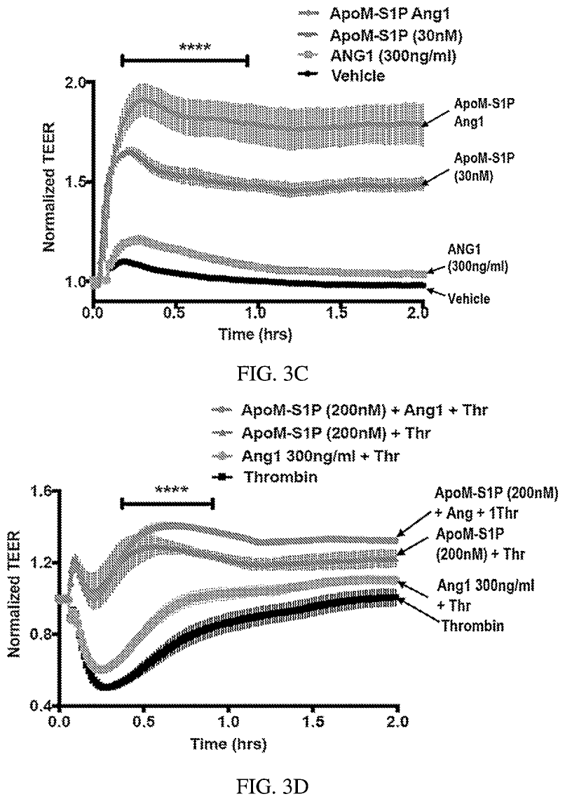

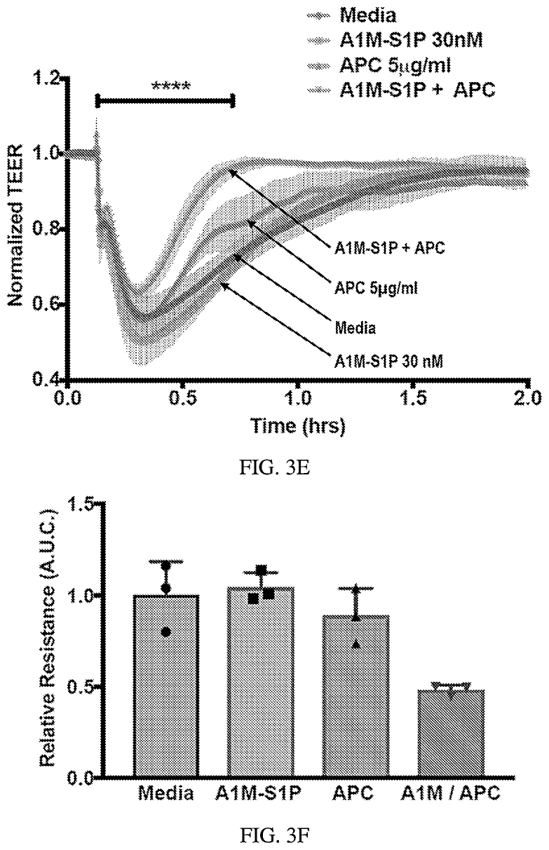

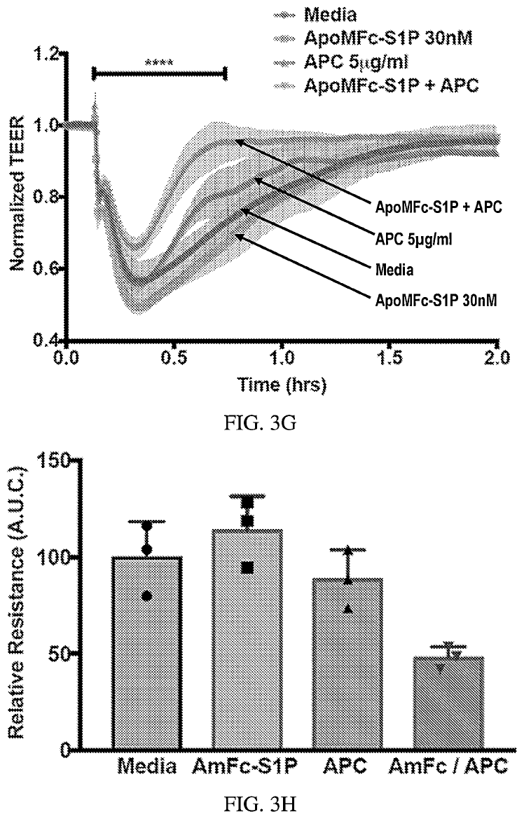

To determine the ability of A1M-S1P regulate, endothelial cell barrier function, transendothelial electrical resistance (TEER) analysis was performed on HUVEC after A1M-S1P stimulation. A dose response study was performed based on S1P content (S1P 10-300 nM; A1M 0.8-24 μg/ml) and strong extended enhancement of endothelial barrier function was observed (FIG. 3A.). As a negative control the maximal dose of native A1M (24 μg/ml) was used, which gave no functional barrier protection. Equivalent enhancement of endothelial barrier function was achieved by A1M-S1P and ApoM-Fc-S1P at very similar EC50 values (˜2.5 g/ml; 30 nM S1P) suggesting the functional equivalency of ApoM as a S1P chaperone whether it is in a soluble or nanodisc form (FIG. 3B). Unloaded A1M and ApoM-Fc did not induce endothelial barrier enhancement.

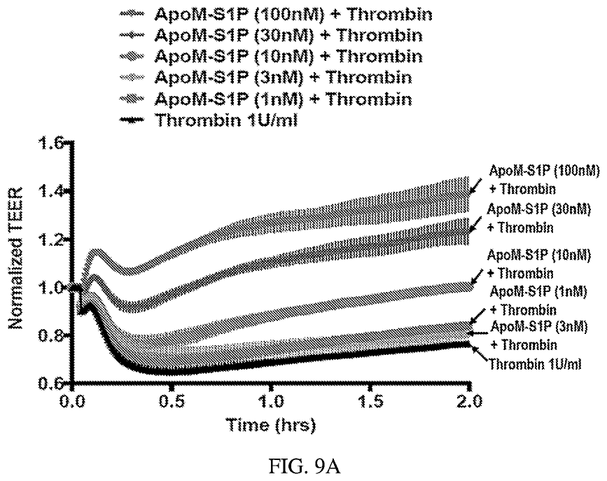

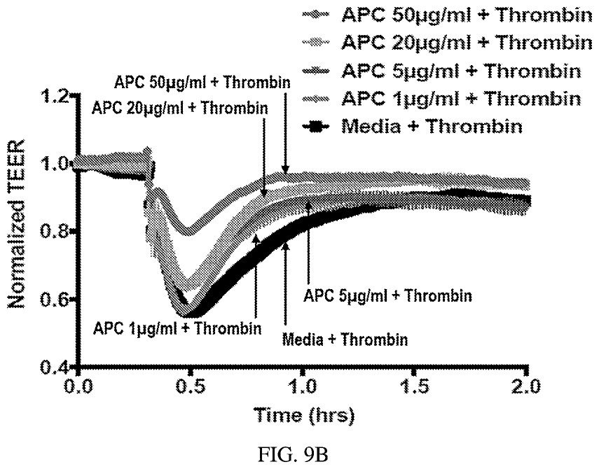

Thrombin activation is a common initiating factor for inflammatory processes and Thrombin is known to degrade endothelial barrier function in vitro and in vivo. It was previously established that activation of the S1PR1 on endothelium inhibits Thrombin-induced barrier degradation (Garcia et al., 2001). A dose response study was performed to evaluate the effect of ApoM-Fc-S1P on Thrombin-induced barrier degradation and confirmed previous observations of S1P-dependent barrier protection against Thrombin and found that S1P enhanced barrier function in a dose-dependent manner (FIG. 9A).

Angiopoietin1 is an additional agent shown to counteract Thrombin activity on endothelium (Pizurki et al., 2003). It was shown that ApoM-Fc-S1P can act in conjunction with Angiopoietin1 to provide enhanced endothelial barrier (FIG. 3C) and observed a clear additive benefit to the combination of both stimuli to block thrombin activity (FIG. 3D). Activated Protein C (APC) is a well-established anti-Thrombin agent (Finigan et al., 2005). APC alone was evaluated in a Thrombin assay by TEER analysis (FIG. 9B) and then in conjunction with both A1M-S1P (FIG. 3E) and Apom-Fc-S1P (FIG. 3G), in all cases using sub-optimal concentrations of both APC (5 μg/ml) and S1P (30 nM). Area under the curve analysis revealed a 51-56% reduction in Thrombin activity for A1M-S1P and a 47-58% reduction in Thrombin activity for ApoM-Fc-S1P. These results reveal a strongly additive effect for both A1M-S1P and ApoM-Fc-S1P with APC, suggesting this combination has utility in blocking the inflammatory effects of Thrombin.

### ApoA1 Attenuates of TNFα-Induced NF-κB Activity and ICAM-1 Expression

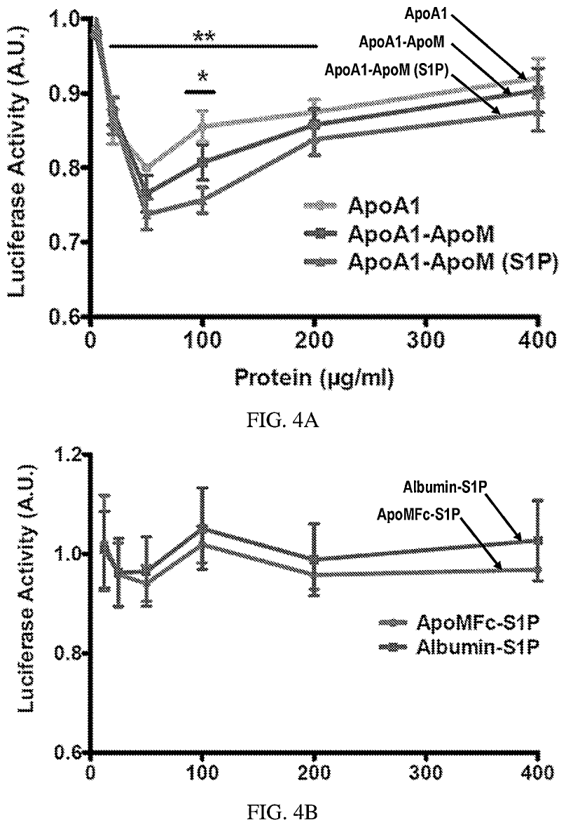

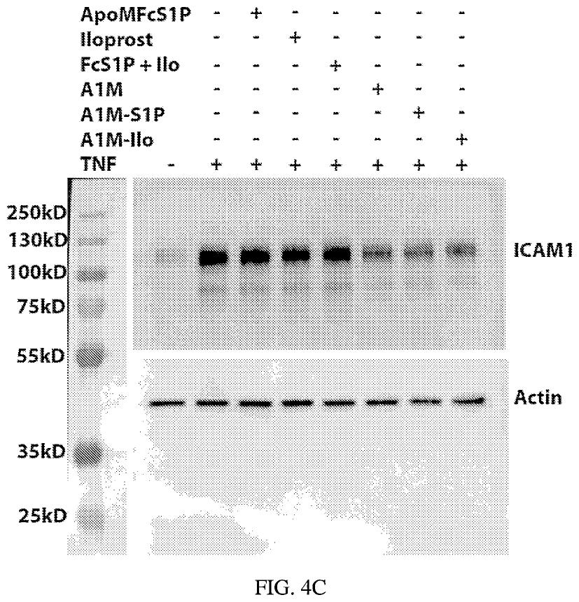

In addition to endothelial barrier protection, HDL-bound S1P attenuates TNFα-induced inflammatory markers such as ICAM-1 adhesion protein expression (Galvani et al., 2015; Cockerill et al., 1995). Albumin-bound S1P was unable to suppress ICAM-1 expression, suggesting that S1PR1 agonism alone is not sufficient to suppress cytokine inflammatory responses. Other studies have suggested that ApoA1 moiety of HDL can engage endothelial cells to induce NO release (Yuhanna et al., 2001) and suppress TLR4- and TNFα-induced NFκB activation in myeloid cells (Fotakis et al., 2019). A luciferase-based reporter assay, was used to evaluate whether ApoA1 or A1M+S1P treatment regulates cytokine-induced NFκB transcription and whether S1PR1 signaling could influence this action. As shown in FIGS. 4A-4C, low doses of ApoA1, A1M and A1M-S1P (25-50 μg/ml) inhibited TNFα-induced NF-κB activity ˜25%. Higher doses (100-400 μg/ml) was not as inhibitory and the dose-response curve exhibited a bi-phasic pattern. In contrast, ApoM-Fc-S1P and albumin-S1P did not inhibit TNFα induced NF-κB activity (FIG. 4B). Previous studies have demonstrated that TNFα induces the adhesion molecule ICAM1 in endothelial cells and that HDL could inhibit this inflammatory process (Cockerill et al., 1995). As shown in FIG. 4C, only ApoA1-containing analytes inhibited ICAM1 expression, independent of S1P or Iloprost content. These data suggest that ApoM-S1P engagement of S1PR1 is not sufficient to attenuate TNFα-induced ICAM1 expression, whereas the ApoA1 moiety of the HDL particle provides an anti-inflammatory function.

A1M Binds to Stable Prostacyclin Analog Iloprost which Cooperates with S1P to Enhance Barrier Function

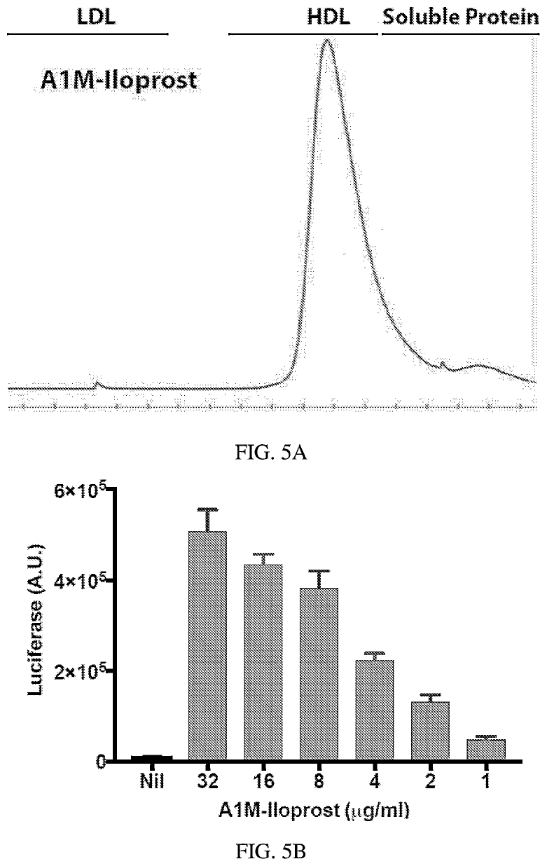

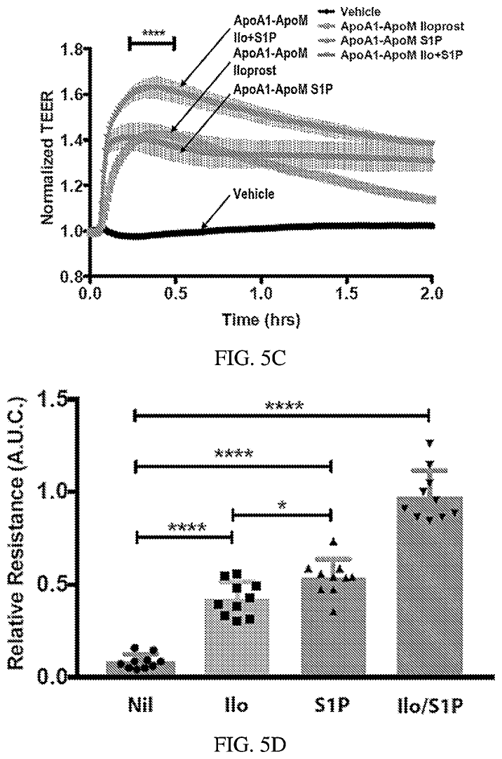

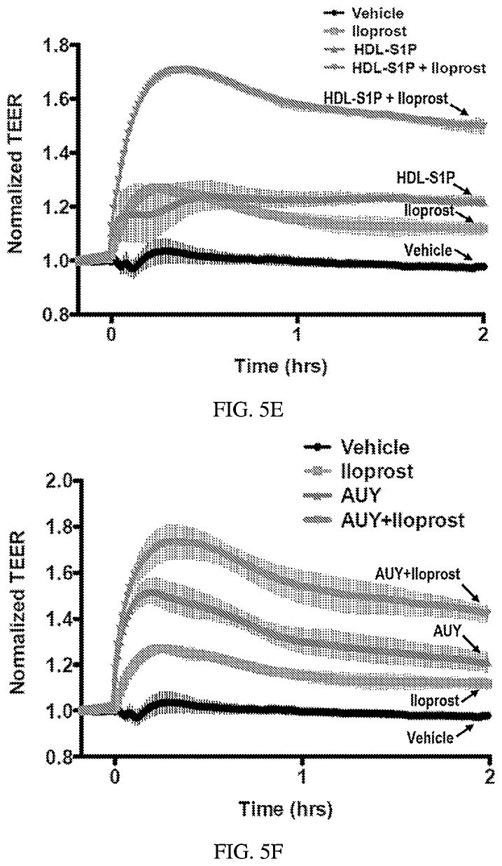

Endothelial cell-derived prostacyclin (PGI₂) acts via its receptor (IP) to activate the Gas/adenylate cyclase/cAMP pathway to suppress platelet aggregation and myeloid inflammatory responses (Reviewed in Pluchart et al., 2017). The lability of PGI₂ due to autohydrolysis is inhibited by HDL association (Morishita et al., 1990). Indeed, it was found that purified A1M nanodiscs can associate stably with Iloprost, a stable PGI₂ analog (Skuballa and Vorbruggen, 1983) (FIG. 5A). Iloprost-bound to ApoA1 was able to activate IP receptor, as determined by cAMP-responsive CREB luciferase reporter assay (FIG. 5B).

Since cAMP/EPAC/Rap1 pathway stimulates endothelial barrier function, it was assessed if A1M nanodisc bound to Iloprost were active in this assay. As shown in FIG. 5C, A1M-Iloprost nanodiscs stimulated endothelial barrier function to a similar degree as A1M-S1P. Combination of A1M-Iloprost and A1M-S1P provided additive barrier promotion of endothelial cells (FIG. 5D). Indeed, Iloprost cooperated with HDL-S1P (FIG. 5E) and the S1PR1-selective agonist AUY954 (FIG. 5F) to enhance endothelial barrier function in an additive manner. These data suggest that HDL stabilization of short-lived endogenous prostanoid PGI₂ may protect the barrier function of the vascular endothelium in a cooperative manner with HDL-S1P.

### A1M-Iloprost and A1M-S1P in Thromboinflammation

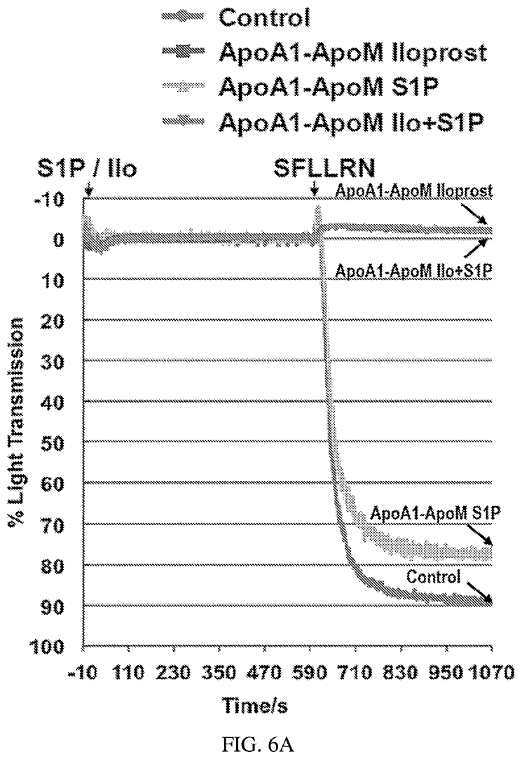

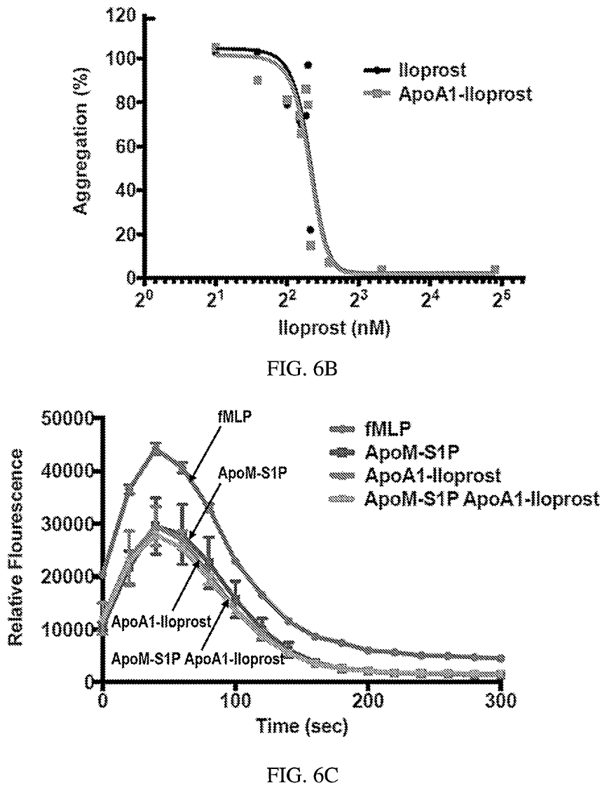

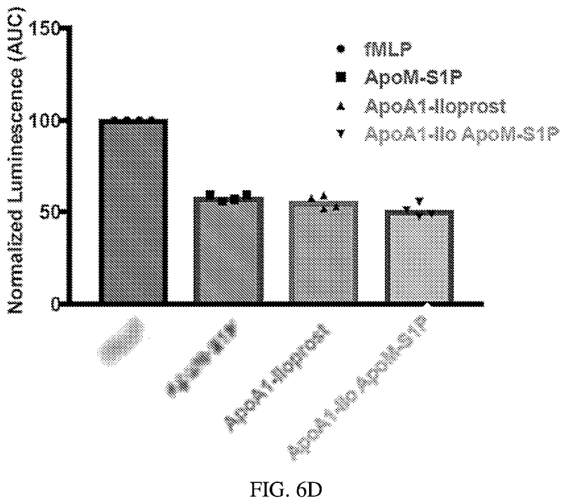

Prostacyclin signaling through the IP receptor inhibits platelet aggregation thus achieving inhibition of thrombosis (Moncada et al., 1977). The effect of A1M-Iloprost and A1M-S1P on human platelet aggregation induced by thrombin receptor activating peptide was evaluated. A1M-Iloprost potently inhibited of platelet aggregation whereas A1M-S1P did not (FIG. 6A). Dose-response studies indicated an EC50 of ˜11 nM for free Iloprost and ˜9 nM for ApoA1 nanodisc-associated-Iloprost (FIG. 6B), suggesting a complete retention of drug potency when Iloprost is associated with HDL-like nanodiscs.

Neutrophil activation results in an oxidative burst, which produces reactive oxygen species (ROS) such as superoxide anion (O2⁻) and hydroxyl radical and singlet oxygen, which are damaging to cells and tissues (Kumar et al., 2019). Thioglycolate-elicited mouse neutrophils were isolated from the mouse and tested their oxidant burst in response to formyl peptide activation. As shown in FIGS. 6C-6D, neutrophil ROS generation was inhibited by both ApoM-S1P (˜42%); ApoA1-Iloprost (˜44%)) co-treatment. Combination treatment did not further decrease ROS formation. The ability of nanodisc-bound S1P and PGI₂ to suppress neutrophil oxidant stress suggests that HDL-bound S1P and PGI₂ may attenuate thromboinflammatory reactions in vivo.

### DISCUSSION

The induction of vascular dysfunction during infectious, traumatic, or chronic metabolic illnesses, and aging has been identified as a primary event for the rise of subsequent tissue and organ pathology. Disrupted and inflamed vasculature are edematous and trigger both platelet driven thrombosis and the subsequent activation of the innate immune response, principally through neutrophils, cumulatively leading to greater organ degradation. Extensive studies of HDL have revealed multiple vascular protective properties that ameliorate these pathological processes (Mineo et al., 2005). Both nascent and lipidated ApoA1, the principal structural protein of HDL, induce reverse cholesterol efflux, which can reduce inflammatory cytokine driven signal transduction (Robert et al., 2021). In addition, HDL was found to induce and chaperone the anti-inflammatory lipid prostacyclin, which acts to inhibit platelet activation and thrombosis, block neutrophil adherence to endothelium, and enhance endothelial barrier function (Riva et al., 1990; Birukova et al, 2013). Finally, recent work identified the HDL associated protein, Apolipoprotein M, as the principal chaperone of the biologically active sphingolipid, S1P, which enhances endothelial homeostasis and barrier function (Christoffersen et al., 2011).

To take advantage of these vascular protective aspects of HDL, a recombinant fusion protein, ApoA1-ApoM (A1M), was made which incorporated the identified anti-inflammatory properties of ApoA1 as well as the barrier protecting properties of S1P. A1M-S1P was demonstrated to activate both Giα and β-arrestin coupling in the three endothelial S1P receptors, S1PR1, S1PR2, and S1PR3 and reproducibly enhance extended endothelial barrier function as judged by TEER analysis, as was observed for other S1P chaperones (Swendeman et al., 2017). Both A1M and A1M-S1P were anti-inflammatory, reducing both TNFα-induced NF-κB activation in a reporter assay and inhibiting downstream inflammatory ICAM1 expression in endothelium. Notably, it was observed that other S1P chaperones ApoM-Fc and albumin had no inhibitory activity, suggesting that the ApoA1 moiety is the primary inhibitor of TNFR signaling.

To further enhance the potential function of A1M, the ApoA1 moiety was lipidated in vitro using standard procedures and it was shown that the stable prostacyclin analogue, Iloprost, can be functionally incorporated into A1M, creating A1M-Iloprost. It was demonstrated that A1M-Iloprost retained the established properties of prostacyclin including activation of PKA-dependent CREB-signaling, and enhancement of Endothelial barrier protection. Intriguingly, in further studies, it was observed that both free and A1M-bound Iloprost could provide additive protection of endothelial barrier function in conjunction with A1M-S1P, suggesting a useful cooperation of these two pathways for endothelial function and protection.

Primary inflammation of endothelium activates multiple pathways that result in further amplification of the inflammatory process including thrombin activation, which both induces disruption of the endothelial barrier and subsequent Platelet activation leading to thrombosis (Mineo et al., 2005). It was demonstrated that A1M-S1P can block Thrombin-induced barrier disruption and enhance the barrier-protective activity of the anti-thrombin, Activated Protein C. Moreover, it was shown that A1M-Iloprost inhibits thrombin-driven platelet aggregation in vitro. A second consequence of endothelial inflammation is Neutrophil recruitment and activation leading to the generation of reactive oxygen species (ROS), a destructive hallmark of inflammation associated with Neutrophils, both in response to external stimuli such as bacterial infection or tissue damage, as well as in response to inflammatory cytokines and chemokines (Pober and Min, 2006, Dimasi et al., 2013). In this regard, the potential utility of A1M-Iloprost/S1P was demonstrated, observing that both lipid ligands potently inhibited the generation of ROS in isolated neutrophils. This approach shows the potential of dampening both platelet-driven thrombosis and neutrophil-dependent tissue damage during tissue inflammation.

These results taken together argue that this recombinant A1M-S1P/Iloprost has the potential to provide a three-pronged approach to recapitulate the established protective effects of HDL by enhancing endothelial homeostasis with S1P, inhibiting inflammatory cytokine signaling with ApoA1, and inhibiting the amplification of inflammation by blocking thrombin activity and both platelet and neutrophil activation. This combined approach has potential utility in many endothelial-associated inflammatory states driven by infectious pathogens, vascular reperfusion injury after trauma or in organ transplantation, as well as more chronic vasculopathies associated with Atherosclerosis, Diabetes and Autoimmunity.

TABLE 1
Sequences

| SEQ ID NO: | Description | Sequence |
|---|---|---|
| 1 | ApoA1 - Human | MKAAVLTLAVLFLTGSQARHFWQQDEPPQSPWDRVKDLATVYVDVLKDS |
| | | GRDYVSQFEGSALGKQLNLKLLDNWDSVTSTFSKLREQLGPVTQEFWDNL |
| | | EKETEGLRQEMSKDLEEVKAKVQPYLDDFQKKWQEEMELYRQKVEPLRAE |
| | | LQEGARQKLHELQEKLSPLGEEMRDRARAHVDALRTHLAPYSDELRQRLAA |
| | | RLEALKENGGARLAEYHAKATEHLSTLSEKAKPALEDLRQGLLPVLESFKVSF |
| | | LSALEEYTKKLNTQ |
| 2 | ApoA1 - Mouse | MKAVVLAVALVFLTGSQAWHVWQQDEPQSQWDKVKDFANVYVDAVKD |
| | | SGRDYVSQFESSSLGQQLNLNLLENWDTLGSTVSQLQERLGPLTRDFWDN |
| | | LEKETDWVRQEMNKDLEEVKQKVQPYLDEFQKKWKEDVELYRQKVAPLG |
| | | AELQESARQKLQELQGRLSPVAEEFRDRMRTHVDSLRTQLAPHSEQMRES |
| | | LAQRLAELKSNPTLNEYHTRAKTHLKTLGEKARPALEDLRHSLMPMLETLKT |
| | | QVQSVIDKASETLTAQ |
| 3 | ApoM - Human | EVHLGQWYFIAGAAPTKEELATFDPVDNIVFNMAAGSAPMQLHLRATIRM |
| | (AA1-20 | KDGLCVPRKWIYHLTEGSTDLRTEGRPDMKTELFSSSCPGGIMLNETGQGY |
| | removed) | QRFLLYNRSPHPPEKCVEEFKSLTSCLDSKAFLLTPRNQEACELSNN |
| 4 | ApoM - Mouse | EPHLGLWYFIAGAAPTTEELATFDPVDNIVFNMAAGSAPRQLQLRATIRTKS |
| | (AA1-20 | GVCVPRKWTYRLTEGKGNMELRTEGRPDMKTDLFSSSCPGGIMLKETGQ |
| | removed) | GYQRFLLYNRSPHPPEKCVEEFKSLTSCLDFKAFLVTPRNQEACPLSSK |
| 5 | ApoM - Human | YQCPEHSQLTTLGVDGKEFPEVHLGQWYFIAGAAPTKEELATFDPVDNIVF |
| | (Full length) | NMAAGSAPMQLHLRATIRMKDGLCVPRKWIYHLTEGSTDLRTEGRPDMK |
| | | TELFSSSCPGGIMLNETGQGYQRFLLYNRSPHPPEKCVEEFKSLTSCLDSKAF |
| | | LLTPRNQEACELSNN |
| 6 | ApoM - Mouse | NQCPEHSQLTALGMDDTETPEPHLGLWYFIAGAAPTTEELATFDPVDNIVF |
| | (Full length) | NMAAGSAPRQLQLRATIRTKSGVCVPRKWTYRLTEGKGNMELRTEGRPD |
| | | MKTDLFSSSCPGGIMLKETGQGYQRFLLYNRSPHPPEKCVEEFKSLTSCLDF |
| | | KAFLVTPRNQEACPLSSK |
| 7 | ApoM - Human | YQCPEHSQLTTLGVDGKEFP |
| | Signal peptide | |
| | (AA1-20) | |
| 8 | ApoM - Mouse | NQCPEHSQLTALGMDDTETP |
| | Signal peptide | |
| | (AA1-20) | |
| 10 | Linker-2 | SGGS |
| 11 | Linker-3 | (SGGS)n |
| 12 | Linker-4 | (GGGS)n |
| 13 | Linker-5 | (GGGGS)n |
| 14 | Linker-6 | (G)n |
| 15 | Linker-7 | (EAAAK)n |
| 16 | Linker-8 | (GGS)n |
| 17 | Linker-9 | SGSETPGTSESATPES |
| 18 | Linker-10 | (XP)n |
| 19 | Linker-11 | SGGSSGSETPGTSESATPESSGGS |
| 20 | Linker-12 | SGGSSGGSSGSETPGTSESATPESSGGSSGGS |
| 21 | Linker-13 | GGSGGSPGSPAGSPTSTEEGTSESATPESGPGTSTEPSEGSAPGSPAGSPTS |
| | | TEEGTSTEPSEGSAPGTSTEPSEGSAPGTSESATPESGPGSEPATSGGSGGS |
| 22 | Linker-14 | GGGGS |
| 23 | ApoA1-ApoM | MKAVVLAVALVFLTGSQAWHVWQQDEPQSQWDKVKDFANVYVDAVKD |
| | fusion protein w/ | SGRDYVSQFESSSLGQQLNLNLLENWDTLGSTVSQLQERLGPLTRDFWDN |
| | purification tag | LEKETDWVRQEMNKDLEEVKQKVQPYLDEFQKKWKEDVELYRQKVAPLG |
| | (FIG. 7B) | AELQESARQKLQELQGRLSPVAEEFRDRMRTHVDSLRTQLAPHSEQMRES |
| | | LAQRLAELKSNPTLNEYHTRAKTHLKTLGEKARPALEDLRHSLMPMLETLKT |
| | | KAQSVIDKASETLTAQTRTRPLGGGGSNQCPEHSQLTALGMDDTETPEPH |
| | | LGLWYFIAGAASTTEELATFDPVDNIVFNMAAGSAPRQLQLRATIRTKSGV |
| | | CVPRKWTYRLTEGKGNMELRTEGRPDMKTDLFSSSCPGGIMLKETGQGY |
| | | QRFLLYNRSPHPPEKCVEEFQSLTSCLDFKAFLVTPRNQEACPLSSKHHHHH |
| | | H |
| 24 | ApoA1-ApoM | MKAVVLAVALVFLTGSQAWHVWQQDEPQSQWDKVKDFANVYVDAVKD |
| | fusion protein | SGRDYVSQFESSSLGQQLNLNLLENWDTLGSTVSQLQERLGPLTRDFWDN |
| | w/o purification | LEKETDWVRQEMNKDLEEVKQKVQPYLDEFQKKWKEDVELYRQKVAPLG |
| | tag | AELQESARQKLQELQGRLSPVAEEFRDRMRTHVDSLRTQLAPHSEQMRES |
| | | LAQRLAELKSNPTLNEYHTRAKTHLKTLGEKARPALEDLRHSLMPMLETLKT |
| | | KAQSVIDKASETLTAQTRTRPLGGGGSNQCPEHSQLTALGMDDTETPEPH |
| | | LGLWYFIAGAASTTEELATFDPVDNIVFNMAAGSAPRQLQLRATIRTKSGV |
| | | CVPRKWTYRLTEGKGNMELRTEGRPDMKTDLFSSSCPGGIMLKETGQGY |
| | | QRFLLYNRSPHPPEKCVEEFQSLTSCLDFKAFLVTPRNQEACPLSSK |
| 25 | ApoA1-ApoM | atgaaagctg tggtgctggc cgtggctctg gtcttcctga cagggagcca |
| | fusion protein | ggcttggcac gtatggcagc aagatgaacc ccagtcccaa tgggacaaag |
| | nucleic acid | tgaaggattt cgctaatgtg tatgtggatg cggtcaaaga cagcggcaga |
| | sequence w/ | gactatgtgt cccagtttga atcctcctcc ttgggccaac agctgaacct gaatctcctg |
| | purification tag | gaaaactggg acactctggg ttcaaccgtt agtcagctgc aggaacggct |
| | (Sequence in FIG. | gggcccattg actcgggact tctgggataa cctggagaaa gaaacagatt |
| | 7A) | gggtgagaca ggagatgaac aaggacctag aggaagtgaa acagaaggtg |
| | | cagccctacc tggacgaatt ccagaagaaa tggaaagagg atgtggagct |
| | | ctaccgccag aaggtggcgc ctctgggcgc cgagctgcag gagagcgcgc |
| | | gccagaagct gcaggagctg caagggagac tgtcccctgt ggctgaggaa |
| | | tttcgcgacc gccatgcgca cacacgtaga ctctctgcgc acacagctag |
| | | cgccccacag cgaacagatg cgcgagagcc tggcccagcg cctggctgag |
| | | ctcaagagca accctacctt gaacgagtac cacaccaggg ccaaaaccca |
| | | cctgaagaca cttggcgaga aagccagacc tgcgctggag gacctgcgcc |
| | | atagtctgat gcccatgctg gagacgctta agaccaaagc ccagagtgtg |
| | | atcgacaagg ccagcgagac tctgactgcc cagacgcgta cgcggccgct |
| | | tggtggaggt ggatctaatc agtgccctga gcacagtcaa ctaactgcgc |
| | | tgggaatgga cgacacagag accccagagc cccacctggg cctgtggtac |
| | | tttattgcgg gagcagcctc caccacggaa gagttggcaa cttttgatcc |
| | | ggtggacaat attgtcttca acatggctgc cggctctgcc ccaaggcagc |
| | | tccagcttcg tgctaccatc cgcacgaaaa gtggggtctg tgtgccccgg |
| | | aagtggacat accgattgac tgaagggaaa ggaaacatgg aactcagaac |
| | | tgaagggcgc ccagacatga aaacagacct gttctccagc tcgtgcccag |
| | | gaggaatcat gctgaaagag acgggccagg gctaccagcg ctttctcctc |
| | | tacaatcggt caccacaccc tccagagaag tgtgtggagg aattccagtc tctgacctct |
| | | tgcttggact tcaaagcctt cttagtgact cccaggaatc aagaggcctg cccgctgtcc |
| | | agcaagcatc accatcacca tcactga |
| 26 | ApoA1-ApoM | atgaaagctg tggtgctggc cgtggctctg gtcttcctga cagggagcca |
| | fusion protein | ggcttggcac gtatggcagc aagatgaacc ccagtcccaa tgggacaaag |
| | nucleic acid | tgaaggattt cgctaatgtg tatgtggatg cggtcaaaga cagcggcaga |
| | sequence w/o | gactatgtgt cccagtttga atcctcctcc ttgggccaac agctgaacct gaatctcctg |
| | purification tag | gaaaactggg acactctggg ttcaaccgtt agtcagctgc aggaacggct |
| | (Sequence in FIG. | gggcccattg actcgggact tctgggataa cctggagaaa gaaacagatt |
| | 7A without | gggtgagaca ggagatgaac aaggacctag aggaagtgaa acagaaggtg |
| | sequence | cagccctacc tggacgaatt ccagaagaaa tggaaagagg atgtggagct |
| | encoding 6X HIS | ctaccgccag aaggtggcgc ctctgggcgc cgagctgcag gagagcgcgc |
| | TAG) | gccagaagct gcaggagctg caagggagac tgtcccctgt ggctgaggaa |
| | | tttcgcgacc gccatgcgca cacacgtaga ctctctgcgc acacagctag |
| | | cgccccacag cgaacagatg cgcgagagcc tggcccagcg cctggctgag |
| | | ctcaagagca accctacctt gaacgagtac cacaccaggg ccaaaaccca |
| | | cctgaagaca cttggcgaga aagccagacc tgcgctggag gacctgcgcc |
| | | atagtctgat gcccatgctg gagacgctta agaccaaagc ccagagtgtg |
| | | atcgacaagg ccagcgagac tctgactgcc cagacgcgta cgcggccgct |
| | | tggtggaggt ggatctaatc agtgccctga gcacagtcaa ctaactgcgc |
| | | tgggaatgga cgacacagag accccagagc cccacctggg cctgtggtac |
| | | tttattgcgg gagcagcctc caccacggaa gagttggcaa cttttgatcc |
| | | ggtggacaat attgtcttca acatggctgc cggctctgcc ccaaggcagc |
| | | tccagcttcg tgctaccatc cgcacgaaaa gtggggtctg tgtgccccgg |
| | | aagtggacat accgattgac tgaagggaaa ggaaacatgg aactcagaac |
| | | tgaagggcgc ccagacatga aaacagacct gttctccagc tcgtgcccag |
| | | gaggaatcat gctgaaagag acgggccagg gctaccagcg ctttctcctc |
| | | tacaatcggt caccacaccc tccagagaag tgtgtggagg aattccagtc |
| | | tctgacctct tgcttggact tcaaagcctt cttagtgact cccaggaatc aagaggcctg |
| | | cccgctgtcc agcaagtga |
| 27 | ApoA1 - Mouse | MKAVVLAVALVFLTGSQAWHVWQQDEPQSQWDKVKDFANVYVDAVKD |
| | (from fusion | SGRDYVSQFESSSLGQQLNLNLLENWDTLGSTVSQLQERLGPLTRDFWDN |
| | protein) | LEKETDWVRQEMNKDLEEVKQKVQPYLDEFQKKWKEDVELYRQKVAPLG |
| | | AELQESARQKLQELQGRLSPVAEEFRDRMRTHVDSLRTQLAPHSEQMRES |
| | | LAQRLAELKSNPTLNEYHTRAKTHLKTLGEKARPALEDLRHSLMPMLETLKT |
| | | KAQSVIDKASETLTAQ |
| 28 | ApoM - Mouse | NQCPEHSQLTALGMDDTETPEPHLGLWYFIAGAASTTEELATFDPVDNIVF |
| | (from fusion | NMAAGSAPRQLQLRATIRTKSGVCVPRKWTYRLTEGKGNMELRTEGRPD |
| | protein) | MKTDLFSSSCPGGIMLKETGQGYQRFLLYNRSPHPPEKCVEEFQSLTSCLDF |
| | | KAFLVTPRNQEACPLSSK |

### Example 1 References

Birukova A A, Wu T, Tian Y, Meliton A, Sarich N, Tian X, Leff A, Birukov K G Iloprost improves endothelial barrier function in lipopolysaccharide-induced lung injury. Eur Respir J. 2013 January; 41(1):165-76. doi: 10.1183/09031936.00148311. Epub 2012 Jul. 12. PMID: 22790920
Blaho V A, Galvani S, Engelbrecht E, Liu C, Swendeman S L, Kono M, Proia R L, Steinman L, Han M H, Hla T. HDL-bound sphingosine-1-phosphate restrains lymphopoiesis and neuroinflammation. Nature. 2015 Jul. 16; 523(7560):342-6. PMID: 26053123
Burg N, Swendeman S, Worgall S, Hla T, Salmon J E Sphingosine 1-Phosphate Receptor 1 Signaling Maintains Endothelial Cell Barrier Function and Protects Against Immune Complex-Induced Vascular Injury. Arthritis Rheumatol. 2018 November; 70(11):1879-1889. PMID: 29781582
Castro P, Palomo M, Moreno-Castano A B, Fernández S, Torramadé-Moix S, Pascual G, Martinez-Sanchez J, Richardson E, Téllez A, Nicolas J M, Carreras E, Richardson P G, Badimon J J, Escolar G, Diaz-Ricart M. Is the Endothelium the Missing Link in the Pathophysiology and Treatment of COVID-19 Complications?Cardiovasc Drugs Ther. 2021 Jun. 7:1-14. PMID: 34097193
Christoffersen C, Nielsen L B, Axler O, Andersson A, Johnsen A H, Dahlback B. Isolation and characterization of human apolipoprotein M-containing lipoproteins. J Lipid Res. 2006 August; 47(8):1833-43. PMID: 16682745
Christoffersen C, Obinata H, Kumaraswamy S B, Galvani S, Ahnström J, Sevvana M, Egerer-Sieber C, Muller Y A, Hla T, Nielsen L B, Dahlback B. Endothelium-protective sphingosine-1-phosphate provided by HDL-associated apolipoprotein M. Proc Natl Acad Sci USA. 2011 Jun. 7; 108(23):9613-8 PMID: 21606363
Cines D. B., Pollak E. S., Buck C. A., Loscalzo J., Zimmerman G. A., McEver R. P., Pober J. S., Wick T. M., Konkle B. A., Schwartz B. S., et al. Endothelial cells in physiology and in the pathophysiology of vascular disorders. Blood. 1998; 91:3527-3561.
Cockerill G W, Rye K A, Gamble J R, Vadas M A, Barter P High-density lipoproteins inhibit cytokine-induced expression of endothelial cell adhesion molecules. J. Arterioscler Thromb Vasc Biol. 1995 November; 15(11):1987-94. PMID: 7583580
Ding B S, Liu C H, Sun Y, Chen Y, Swendeman S L, Jung B, Chavez D, Cao Z, Christoffersen C, Nielsen L B, Schwab S R, Rafii S, Hla T. HDL activation of endothelial sphingosine-1-phosphate receptor-1 (S1P(1)) promotes regeneration and suppresses fibrosis in the liver. JCI Insight. 2016 Dec. 22; 1(21) PMID: 28018969
Ding B S, Yang D, Swendeman S L, Christoffersen C, Nielsen L B, Friedman S L, Powell C A, Hla T, Cao Z. Aging Suppresses Sphingosine-1-Phosphate Chaperone ApoM in Circulation Resulting in Maladaptive Organ Repair. Dev Cell. 2020 Jun. 22; 53(6):677-690. PMID: 32544390
Engelbrecht E, Levesque M V, He L, Vanlandewijck M, Nitzsche A, Niazi H, Kuo A, Singh S A, Aikawa M, Holton K, Proia R L, Kono M, Pu W T, Camerer E, Betsholtz C, Hla T. Sphingosine 1-phosphate-regulated transcriptomes in heterogenous arterial and lymphatic endothelium of the aorta. Elife. 2020 Feb. 24; 9:e52690.
Esper R J, Nordaby R A, Vilariño J O, Paragano A, Cacharrón J L, Machado R A. Endothelial dysfunction: a comprehensive appraisal. Cardiovasc Diabetol. 2006 Feb. 23; 5:4. PMID: 16504104
Finigan J H, Dudek S M, Singleton P A, Chiang E T, Jacobson J R, Camp S M, Ye S Q, Garcia J G. Activated protein C mediates novel lung endothelial barrier enhancement: role of sphingosine 1-phosphate receptor transactivation. J Biol Chem. 2005 Apr. 29; 280(17):17286-93. PMID: 15710622
Fotakis P, Kothari V, Thomas D G, Westerterp M, Molusky M M, Altin E, Abramowicz S, Wang N, He Y, Heinecke J W, Bornfeldt K E, Tall A R. Anti-Inflammatory Effects of HDL (High-Density Lipoprotein) in Macrophages Predominate Over Proinflammatory Effects in Atherosclerotic Plaques. Arterioscler Thromb Vasc Biol. 2019 December; 39(12):e253-e272 PMID: 3157808
Dimasi D, Sun W Y, Bonder C S. Neutrophil interactions with the vascular endothelium. Int Immunopharmacol. 2013 December; 17(4):1167-75. PMID: 23863858
Galvani S, Sanson M, Blaho V A, Swendeman S L, Obinata H, Conger H, Dahlback B, Kono M, Proia R L, Smith J D, Hla T. HDL-bound sphingosine 1-phosphate acts as a biased agonist for the endothelial cell receptor S1P1 to limit vascular inflammation Sci Signal. 2015 Aug. 11; 8(389) PMID: 26268607
Garcia J G, Liu F, Verin A D, Birukova A, Dechert M A, Gerthoffer W T, Bamberg J R, English D. Sphingosine 1-phosphate promotes endothelial cell barrier integrity by Edg-dependent cytoskeletal rearrangement. J Clin Invest. 2001; 108:689-701.
Heinecke J W. The protein cargo of HDL: implications for vascular wall biology and therapeutics. J Clin Lipidol. 2010 September-October; 4(5):371-5 PMID: 20975842
Hisano Y, Kono M, Cartier A, Engelbrecht E, Kano K, Kawakami K, Xiong Y, Piao W, Galvani S, Yanagida K, Kuo A, Ono Y, Ishida S, Aoki J, Proia R L, Bromberg J S, Inoue A, Hla T. Lysolipid receptor cross-talk regulates lymphatic endothelial junctions in lymph nodes. J Exp Med. 2019 Jul. 1; 216(7):1582-1598. PMID: 31147448
Inoue A, Raimondi F, Kadji F M N, Singh G, Kishi T, Uwamizu A, Ono Y, Shinjo Y, Ishida S, Arang N, Kawakami K, Gutkind J S, Aoki J, Russell R B. Illuminating G-Protein-Coupling Selectivity of GPCRs. Cell. 2019 Jun. 13; 177(7):1933-1947. PMID: 31160049
Kumar S, Dikshit M Metabolic Insight of Neutrophils in Health and Disease Front Immunol. 2019; 10: 2099. PMC6764236
Lepley D, Paik J-H, Hla T, Ferrer F. The G protein-coupled receptor S1P2 regulates Rho/Rho kinase pathway to inhibit tumor cell migration Cancer Res. 2005 May 1; 65(9):3788-95. PMID: 15867375
Mineo C, Deguchi H, Griffin J H, Shaul P W. Endothelial and antithrombotic actions of HDL. Circ Res. 2006 Jun. 9; 98(11):1352-64. PMID: 16763172
Moncada S, Higgs E A, Vane J R. Human arterial and venous tissues generate prostacyclin (prostaglandin x), a potent inhibitor of platelet aggregation. Lancet. 1977 Jan. 1; 1(8001):18-20. PMID: 63657
Morishita H, Yui Y, Hattori R, Aoyama T, Kawai C. Increased hydrolysis of cholesteryl ester with prostacyclin is potentiated by high density lipoprotein through the prostacyclin stabilization. J Clin Invest. 1990 December; 86(6):1885-91. PMID: 2174909
Obinata H, Hla T. Int Immunol. 2019 Aug. 23; 31(9):617-625. Sphingosine 1-phosphate and inflammation PMID: 31049553
Oda M N, Hargreaves P L, Beckstead J A, Redmond K A, van Antwerpen R, Ryan R O. Reconstituted high density lipoprotein enriched with the polyene antibiotic amphotericin B. J Lipid Res. 2006 February; 47(2):260-7. PMID: 16314670
Ouimet M, Barrett T J, Fisher E A HDL and Reverse Cholesterol transport: Basic Mechanisms and their Roles in Vascular Health and Disease. Circ Res. 2019 May 10; 124(10): 1505-1518. PMC6813799
Pizurki L, Zhou Z, Glynos K, Roussos C, Papapetropoulos A. Angiopoietin-1 inhibits endothelial permeability, neutrophil adherence and IL-8 production. Br J Pharmacol. 2003 May; 139(2):329-36. PMID: 12770938
Pluchart H, Khouri C, Blaise S, Roustit M, Cracowski J L. Targeting the Prostacyclin Pathway: Beyond Pulmonary Arterial Hypertension. Trends Pharmacol Sci. 2017 June; 38(6):512-523 PMID: 28412042
Pober J S, Min W. Endothelial cell dysfunction, injury and death. Handb Exp Pharmacol. 2006; (176 Pt 2):135-56. PMID: 16999227
Pober J S, Sessa W C. Evolving functions of endothelial cells in inflammation. Nat. Rev. Immunol. 2007; 7:803-815. [PubMed: 17893694]
Proia R L, Hla T. Emerging biology of sphingosine-1-phosphate: its role in pathogenesis and therapy. J Clin Invest. 2015 April; 125(4):1379-87 PMID: 25831442
Iloprost inhibits neutrophil-induced lung injury and neutrophil adherence to endothelial monolayers.
Riva C M, Morganroth M L, Ljungman A G, Schoeneich S O, Marks R M, Todd R F 3rd, Ward P A, Boxer L A. Am J Respir Cell Mol Biol. 1990 October; 3(4):301-9. doi: 10.1165/ajrcmb/3.4.301. PMID: 1698399
Robert J, Osto, E, von Eckardstein, A The Endothelium is both a target and a barrier of HDL's proetective Functions Cells 2021 Apr. 28; 10(5)1041. PMID: 33924941
Ruiz M, Frej C, Holmdr A, Guo L J, Tran S, Dahlback B. High-Density Lipoprotein-Associated Apolipoprotein M Limits Endothelial Inflammation by Delivering Sphingosine-1-Phosphate to the Sphingosine-1-Phosphate Receptor 1. Arterioscler Thromb Vasc Biol. 2017 January; 37(1):118-129. PMID: 27879252
Schwendeman A, Sviridov D O, Yuan W, Guo Y, Morin E E, Yuan Y, Stonik J, Freeman L, Ossoli A, Thacker S, Killion S, Pryor M, Chen Y E, Turner S, Remaley A T. The effect of phospholipid composition of reconstituted HDL on its cholesterol efflux and anti-inflammatory properties. J Lipid Res. 2015 September; 56(9):1727-37. PMID: 26117661
Skuballa W, Vorbruggen H. Synthesis of ciloprost (ZK 36 374): a chemically stable and biologically potent prostacyclin analog. Adv Prostaglandin Thromboxane Leukot Res. 1983; 11:299-305. PMID: 6189381
Swendeman S L, Xiong Y, Cantalupo A, Yuan H, Burg N, Hisano Y, Cartier A, Liu C H, Engelbrecht E, Blaho V, Zhang Y, Yanagida K, Galvani S, Obinata H, Salmon J E, Sanchez T, Di Lorenzo A, Hla T. An engineered S1P chaperone attenuates hypertension and ischemic injury. Sci Signal. 2017 Aug. 15; 10(492) PMID: 28811382
Suzuki M, Pritchard D K, Becker L, Hoofnagle A N, Tanimura N, Bammler T K, Beyer R P, Bumgarner R, Vaisar T, de Beer M C, de Beer F C, Miyake K, Oram J F, Heinecke J W. High-density lipoprotein suppresses the type i interferon response, a family of potent antiviral immunoregulators, in macrophages challenged with lipopolysaccharide. Circulation. 2010; 122:1919-1927
Trinh R, Gurbaxani B, Morrison S L, Seyfzadeh M. Optimization of codon pair use within the (GGGGS)3 linker sequence results in enhanced protein expression. Mol Immunol. 2004 January; 40(10):717-22 PMID: 14644097
Velagapudi S, Rohrer L, Poti F, Feuerborn R, Perisa D, Wang D, Panteloglou G, Potapenko A, Yalcinkaya M, Hulsmeier A J, Hesse B, Lukasz A, Liu M, Parks J S, Christoffersen C, Stoffel M, Simoni M, Nofer J R, von Eckardstein A. Apolipoprotein M and Sphingosine-1-Phosphate Receptor 1 Promote the Transendothelial Transport of High-Density Lipoprotein. Arterioscler Thromb Vasc Biol. 2021 Aug. 19. PMID: 34407633
Yuhanna I S, Zhu Y, Cox B E, Hahner L D, Osborne-Lawrence S, Lu P, Marcel Y L, Anderson R G, Mendelsohn M E, Hobbs H H, Shaul P W. High-density lipoprotein binding to scavenger receptor-BI activates endothelial nitric oxide synthase. Nat Med. 2001 July; 7(7):853-7. PMID: 11433352
Yvan-Charvet L, Wang N, Tall A R. The role of HDL, ABCA1 and ABCG1 transporters in cholesterol efflux an sc Biol. 2010 February; 30(2): 139-143. PMC2812788

What is claimed is:

1. A fusion protein comprising ApoA1 and ApoM.

2. The fusion protein of claim 1, wherein ApoA1 comprises an amino acid sequence that is 90% identical to any one of SEQ ID NOs: 1-2 or 27, optionally wherein the ApoA1 comprises the amino acid sequence any one of SEQ ID NOs: 1-2 or 27.

3. The fusion protein of claim 1 or claim 2, wherein ApoM comprises an amino acid sequence that is 90% identical to any one of SEQ ID NOs: 3-4 or 28, optionally wherein the ApoA1 comprises the amino acid sequence of any one of SEQ ID NOs: 3-4 or 28.

4. The fusion protein of any one of claims 1-3, wherein the ApoA1 is fused to the N-terminus of ApoM.

5. The fusion protein of any one of claims 1-4, wherein the ApoA1 is fused to the C-terminus of ApoM.

6. The fusion protein of any one of claims 1-5, wherein the ApoA1 and ApoM are fused via a linker, optionally wherein the linker is a peptide linker.

7. The fusion protein of claim 6, wherein the linker comprises the amino acid sequence of SEQ ID NO: 22.

8. The fusion protein of any one of claims 1-7, comprising an amino acid sequence that is 90% identical to any one of SEQ ID NOs: 23-24, optionally wherein the fusion protein comprises the amino acid sequence of any one of SEQ ID NOs: 23-24.

9. A nucleic acid molecule comprising a polynucleotide sequence encoding the fusion protein of any one of claims 1-8.

10. The nucleic acid molecule of claim 9, wherein the polynucleotide sequence comprises a nucleotide sequence that is at least 90% identical to SEQ ID NO: 25 or 26, optionally wherein the polynucleotide sequence comprises the nucleotide sequence of SEQ ID NO: 25 or 26.

11. The nucleic acid molecule of claim 9 or claim 10, wherein the polynucleotide sequence is operably linked to a promoter.

12. A construct comprising the nucleic acid molecule of any one of claims 9-11.

13. The construct of claim 12, wherein the construct is a plasmid or vector, optionally a viral vector.

14. A cell comprising the fusion protein of any one of claims 1-8, the nucleic acid sequence of any one of claims 9-11, or the construct of any one of claims 12-13.

15. The cell of claim 14, wherein the cell is a prokaryotic cell.

16. The cell of claim 14, wherein the cell is a eukaryotic cell, optionally a human cell.

17. A lipoprotein comprising the fusion protein of any one of claims 1-8 and a lipid.

18. The lipoprotein of claim 17, wherein the lipoprotein is a S1P receptor agonist or antagonist, or a prostaglandin agonist or antagonist.

19. The lipoprotein of claim 17 or claim 18, wherein the lipid is selected from the group consisting of a prostaglandin, sphingosine 1-phosphate (S1P), a leukotriene, phosphatidyl choline.

20. The lipoprotein of claim 19, wherein the lipid is Iloprost.

21. The lipoprotein of claim 19, wherein the lipid is sphingosine-1-phosphate.

22. The lipoprotein of any one of claims 17-21, wherein the lipoprotein is non-covalently bound to the lipid.

23. The lipoprotein of any one of claims 17-21, wherein the lipoprotein is covalently bound to the lipid.

24. The lipoprotein of any one of claims 17-23, wherein the lipoprotein is incorporated into a nanoparticle.

25. The nanoparticle of claim 24, wherein the nanoparticle is a nanodisk.

26. The nanoparticle of claim 24 or claim 25, wherein the nanoparticle is 70% unlipidated fusion protein and 30% lipoprotein.

27. A method of treating a subject having a disease associated with vascular endothelial dysfunction, comprising administering the fusion protein of any one of claims 1-8 or the lipoprotein of any one of claims 17-26.

28. The method of claim 27, wherein the disease is selected from the group consisting of thrombosis, or thrombotic inflammation.

29. The method of claim 27 and 28, wherein the thrombotic inflammation is associated with cardiovascular disease, cerebrovascular disease, diabetes, atherosclerosis, an autoimmune syndrome or chronic inflammatory diseases.

30. A method of reducing inflammation in a subject, comprising administering a drug selected from the group consisting of the fusion protein of any one of claims 1-8, or the lipoprotein of any one of claims 17-26.

31. The method of any one of claims 27-30, wherein the drug is administered in a therapeutically effective amount.

32. The method of claim 30 or claim 31, wherein the inflammation is associated with TNFalpha-induced NF-kappaB activation.

33. The method of any one of claims 30-32, wherein the inflammation is associated with cardiovascular disease, cerebrovascular disease, diabetes, atherosclerosis, an autoimmune syndrome or chronic inflammatory diseases.

34. The method of any one of claims 27-33, wherein the disease is selected from the group consisting of diabetic nephropathy, lupus, and COVID-19 syndrome induced blood clots.

35. The method of claim 34, further comprising administrating an anti-thrombin agent.

36. The method of any one of claim 35, wherein the anti-thrombin agent is Angiopoietin1 or Activated Protein C (APC).
